SEQUENCE LISTING (149 sequences)

SEQ ID NO: 1 - protein, 511 aa, Vesicular stomatitis Indiana virus
  VSIV-G
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 2 - protein, 517 aa, Vesicular stomatitis New Jersey virus
  VSNJV-G
MLSYLIFALVVSPILGKIEIVFPQHTTGDWKRVPHEYNYCPTSADKNSHGTQTGIPVELTMPKGLTTHQVDGFMCHSALWMTTCDFRWYGPKYITHSIHNEEPTDYQCLEAIKAYKDGVSFNPGFPPQSCGYGTVTDAEAHIVTVTPHSVKVDEYTGEWIDPHFIGGRCKGQICETVHNSTKWFTSSDGESVCSQLFTLVGGTFFSDSEEITSMGLPETGIRSNYFPYVSTEGICKMPFCRKPGYKLKNDLWFQITDPDLDKTVRDLPHIKDCDLSSSIVTPGEHATDISLISDVERILDYALCQNTWSKIEAGEPITPVDLSYLGPKNPGAGPVFTIINGSLHYFMSKYLRVELESPVIPRMEGKVAGTRIVRQLWDQWFPFGEVEIGPNGVLKTKQGYKFPLHIIGTGEVDNDIKMERIVKHWEHPHIEAAQTFLKKDDTEEVLYYGDTGVSKNPVELVEGWFSGWRSSIMGVLAVIIGFVILIFLIRLIGVLSSLFRQKRRPIYKSDVEMAHFR

SEQ ID NO: 3 - protein, 530 aa, Chandipura virus
  CHPV-G
MLSQVTLQAFVVMCLIYRAYSSLSIAFPESTKLDWKPVTKNTRYCPMGGEWFLEPGLQEESFLSSTPIGATPSKSDGFLCHAAKWVTTCDFRWYGPKYITHSIHNIKPTRSDCDSALASYKSGTLINPGFPPDSCGYASVTDSEFLVIMITPHHVGVDDYRGHWVDPLFVGGECDQSYCDTIHNSSVWIPADTTKKNICGQSFTPLTVTVAYDKTKDISAGALVFKSKYHSHMEGARTCRLTYCGRTGIKFPNGEWVSLDIKTKIQDKQLLPLFKECPAGTEVRSTLQSDGAQVLTSEIQRLLDYSLCQNTWEKVDRKEPLSPLDLSYLASKSPGKGLAYTVINGTLSFAHTRYVRMWIDGPVLKELKGKRESASGIASDIWTQWFKYGDMEIGPNGLLKTKSGYKFPWHLIGMGIVDNELHELSEANPLDHPQLPHAQSIADDSEEIFFGDTGVSKNPVELVTGWFTSWKESLAAGVVLILTVVLIYGVLRCFPVLCMPCRKTKWKKEVERSDSFEMRIFKPNNMRARV

SEQ ID NO: 4 - protein, 512 aa, Cocal virus
  COCV-G
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 5 - protein, 529 aa, Piry virus
  PIRYV-G
MDLFPILVVVLMTDTVLGKFQIVFPDQNELEWRPVVGDSRHCPQSSEMQFDGSRSQTILTGKAPVGITPSKSDGFICHAAKWVTTCDFRWYGPKYITHSIHHLRPTTSDCETALQRYKDGSLINLGFPPESCGYATVTDSEAMLVQVTPHHVGVDDYRGHWIDPLFPGGECSTNFCDTVHNSSVWIPKSQKTDICAQSFKNIKMTASYPSEGALVSDRFAFHSAYHPNMPGSTVCIMDFCEQKGLRFTNGEWMGLNVEQSIREKKISAIFPNCVAGTEIRATLESEGARTLTWETQRMLDYSLCQNTWDKVSRKEPLSPLDLSYLSPRAPGKGMAYTVINGTLHSAHAKYIRTWIDYGEMKEIKGGRGEYSKAPELLWSQWFDFGPFKIGPNGLLHTGKTFKFPLYLIGAGIIDEDLHELDEAAPIDHPQMPDAKSVLPEDEEIFFGDTGVSKNPIELIQGWFSNWRESVMAIVGIVLLIVVTFLAIKTVRVLNCLWRPRKKRIVRQEVDVESRLNHFEMRGFPEYVKR

SEQ ID NO: 6 - protein, 523 aa, Isfahan virus
  ISFV-G
MTSVLFMVGVLLGAFGSTHCSIQIVFPSETKLVWKPVLKGTRYCPQSAELNLEPDLKTMAFDSKVPIGITPSNSDGYLCHAAKWVTTCDFRWYGPKYITHSVHSLRPTVSDCKAAVEAYNAGTLMYPGFPPESCGYASITDSEFYVMLVTPHPVGVDDYRGHWVDPLFPTSECNSNFCETVHNATMWIPKDLKTHDVCSQDFQTIRVSVMYPQTKPTKGADLTLKSKFHAHMKGDRVCKMKFCNKNGLRLGNGEWIEVGDEVMLDNSKLLSLFPDCLVGSVVKSTLLSEGVQTALWETDRLLDYSLCQNTWEKIDRKEPLSAVDLSYLAPRSPGKGMAYIVANGSLMSAPARYIRVWIDSPILKEIKGKKESASGIDTVLWEQWLPFNGMELGPNGLIKTKSGYKFPLYLLGMGIVDQDLQELSSVNPVDHPHVPIAQAFVSEGEEVFFGDTGVSKNPIELISGWFSDWKETAAALGFAAISVILIIGLMRLLPLLCRRRKQKKVIYKDVELNSFDPRQAFHR

SEQ ID NO: 7 - protein, 509 aa, Spring viraemia of carp virus
  SVCV-G
MSIISYIAFLLLIDSTLGIPIFVPSGQNISWQPVIQPFDYQCPIHGNLPNTMGLSATKLTIKSPSVFSTDKVSGWICHAAEWKTTCDYRWYGPQYITHSIHPISPTIDECKRIISRIASGTDEDLGFPPQSCGWASVTTVSNTNYKVVPHSVHLEPYGGHWIDHEFNGGECREKVCEMKGNHSIWITDETVQHECEKHIEEVEGIMYGNAPRGDALYINNFIIDKHHRVYRFGGSCRMKFCDKDGIKFTRGDWVEKTAETLTKIYANTPECADGTLVSGHRPGLDLIDTVFNLENVVEYTLCEGTKRKINEQEKLTSVDLSYLAPRIGGFGSVFRVRNGTLERGSTTYIRIEVEGPIVDSLTGTDPRTNASRVFWDDWELDGNIYQGFNGVYKGKDGKIHIPLNMIESGIIDDELQHAFQADIIPHPHYDDDEIREDDIFFDNTGENGNPVDAVVEWVSGWGTSLKFFGTTLVALILIFLLIRCCVACTYLMKKSKRPATESHEMRSLV

SEQ ID NO: 8 - protein, 511 aa, Vesicular stomatitis Alagoas virus
  VSAV-G
MTPAFILCMLLAGSSWAKFTIVFPQSQKGDWKDVPPNYRYCPSSADQNWHGDLLGVNIRAKMPKVHKAIKADGWMCHAAKWVTTCDYRWYGPQYITHSIHSFIPTKAQCEESIKQTKEGVWINPGFPPKNCGYASVSDAESIIVQATAHSVMIDEYSGDWLDSQFPTGRCTGSTCETIHNSTLWYADYQVTGLCDSALVSTEVTFYSEDGLMTSIGRQNTGYRSNYFPYEKGAAACRMKYCTHEGIRLPSGVWFEMVDKELLESVQMPECPAGLTISAPTQTSVDVSLILDVERMLDYSLCQETWSKVHSGLPISPVDLGYIAPKNPGAGPAFTIVNGTLKYFDTRYLRIDIEGPVLKKMTGKVSGTPTKRELWTEWFPYDDVEIGPNGVLKTPEGYKFPLYMIGHGLLDSDLQKTSQAEVFHHPQIAEAVQKLPDDETLFFGDTGISKNPVEVIEGWFSNWRSSVMAIVFAILLLVITVLMVRLCVAFRHFCCQKRHKIYNDLEMNQLRR

SEQ ID NO: 9 - protein, 523 aa, Carajas virus
  CJSV-G
MKMKMVIAGLILCIGILPAIGKITISFPQSLKGDWRPVPKGYNYCPTSADKNLHGDLIDIGLRLRAPKSFKGISADGWMCHAARWITTCDFRWYGPKYITHSIHSFRPSNDQCKEAIRLTNEGNWINPGFPPQSCGYASVTDSESVVVTVTKHQVLVDEYSGSWIDSQFPGGSCTSPICDTVHNSTLWHADHTLDSICDQEFVAMDAVLFTESGKFEEFGKPNSGIRSNYFPYESLKDVCQMDFCKRKGFKLPSGVWFEIEDAEKSHKAQVELKIKRCPHGAVISAPNQNAADINLIMDVERILDYSLCQATWSKIQNKEALTPIDISYLGPKNPGPGPAFTIINGTLHYFNTRYIRVDIAGPVTKEITGFVSGTSTSRVLWDQWFPYGENSIGPNGLLKTASGYKYPLFMVGTGVLDADIHKLGEATVIEHPHAKEAQKVVDDSEVIFFGDTGVSKNPVEVVEGWFSGWRSSLMSIFGIILLIVCLVLIVRILIALKYCCVRHKKRTIYKEDLEMGRIPRRA

SEQ ID NO: 10 - protein, 512 aa, Maraba virus
  MARAV-G
MLRLFLFCFLALGAHSKFTIVFPHHQKGNWKNVPSTYHYCPSSSDQNWHNDLTGVSLHVKIPKSHKAIQADGWMCHAAKWVTTCDFRWYGPKYITHSIHSMSPTLEQCKTSIEQTKQGVWINPGFPPQSCGYATVTDAEVVVVQATPHHVLVDEYTGEWIDSQLVGGKCSKEVCQTVHNSTVWHADYKITGLCESNLASVDITFFSEDGQKTSLGKPNTGFRSNHFAYESGEKACRMQYCTQWGIRLPSGVWFELVDKDLFQAAKLPECPRGSSISAPSQTSVDVSLIQDVERILDYSLCQETWSKIRAKLPVSPVDLSYLAPKNPGSGPAFTIINGTLKYFETRYIRVDISNPIIPHMVGTMSGTTTERELWNDWYPYEDVEIGPNGVLKTPTGFKFPLYMIGHGMLDSDLHKSSQAQVFEHPHAKDAASQLPDDETLFFGDTGLSKNPVELVEGWFSSWKSTLASFFLIIGLGVALIFIIRIIVAIRYKYKGRKTQKIYNDVEMSRLGNK

SEQ ID NO: 11 - protein, 16 aa, Artificial Sequence
  Signal peptide of VSV-G
MKCLLYLAFLFIGVNC

SEQ ID NO: 12 - protein, 17 aa, Artificial Sequence
  Signal peptide of Gaussia luciferase
MGVKVLFALICIAVAEA

SEQ ID NO: 13 - protein, 22 aa, Artificial Sequence
  Signal peptide of Mus musculus Ig kappa light chain precursor (mutant A2)
MDMRAPAGIFGFLLVLFPGYRS

SEQ ID NO: 14 - protein, 18 aa, Artificial Sequence
  Signal peptide of Homo sapiens serum albumin preproprotein
MKWVTFISLLFLFSSAYS

SEQ ID NO: 15 - protein, 19 aa, Artificial Sequence
  Signal peptide of Homo sapiens immunoglobulin heavy chain
MDWTWRVFCLLAVTPGAHP

SEQ ID NO: 16 - protein, 19 aa, Artificial Sequence
  Signal peptide of Homo sapiens immunoglobulin light chain
MAWSPLFLTLITHCAGSWA

SEQ ID NO: 17 - protein, 19 aa, Artificial Sequence
  Signal peptide of Homo sapiens azurocidin preproprotein
MTRLTVLALLAGLLASSRA

SEQ ID NO: 18 - protein, 20 aa, Artificial Sequence
  Signal peptide of Homo sapiens Cystatin-S precursor
MARPLCTLLLLMATLAGALA

SEQ ID NO: 19 - protein, 15 aa, Artificial Sequence
  Signal peptide of Pseudopleuronectes americanus trypsinogen 2 precursor
MRSLVFVLLIGAAFA

SEQ ID NO: 20 - protein, 22 aa, Artificial Sequence
  Signal peptide of Mesobuthus martensii potassium channel blocker
MSRLFVFILIALFLSAIIDVMS

SEQ ID NO: 21 - protein, 21 aa, Artificial Sequence
  Signal peptide of Conus leopardus N1-conotoxin lp1.3
MGMRMMFIMFMLVVLATTVVS

SEQ ID NO: 22 - protein, 18 aa, Artificial Sequence
  Signal peptide of Saccharomyces cerevisiae N1-galactosidase (mutant m3)
MRAFLFLTACISLPGVFG

SEQ ID NO: 23 - protein, 18 aa, Artificial Sequence
  Signal peptide of Aspergillus niger cellulase
MKFQSTLLLAAAAGSALA

SEQ ID NO: 24 - protein, 24 aa, Artificial Sequence
  Signal peptide of Nepenthes gracilis aspartic proteinase nepenthesin-1
MASSLYSFLLALSIVYIFVAPTHS

SEQ ID NO: 25 - protein, 26 aa, Artificial Sequence
  Signal peptide of Nepenthes rafflesiana acid chitinase
MKTHYSSAILPILTLFVFLSINPSHG

SEQ ID NO: 26 - protein, 36 aa, Artificial Sequence
  Signal peptide of M28 virus K28 prepro-toxin
MESVSSLFNIFSTIMVNYKSLVLALLSVSNLKYARG

SEQ ID NO: 27 - protein, 21 aa, Artificial Sequence
  Signal peptide of Zygosaccharomyces bailii killer toxin zygocin precursor
MKAAQILTASIVSLLPIYTSA

SEQ ID NO: 28 - protein, 19 aa, Artificial Sequence
  Signal peptide of Vibrio cholerae O139 cholera toxin
MIKLKFGVFFTVLLSSAYA

SEQ ID NO: 29 - protein, 50 aa, Artificial Sequence
  Signal peptide of Saccharomyces cerevisiae- derived adhesion subunit of N1-agglutinin
MQLLRCFSIFSVIASVLAQELTTICEQIPSPTLESTPYSLSTTTILANGK

SEQ ID NO: 30 - protein, 51 aa, Artificial Sequence
  Signal peptide of Saccharomyces cerevisiae- derived exo-1,3-N2 glucanase
MLSLKTLLCTLLTVSSVLATPVPARDPSSIQFVHEENKKRYYDYDHGSLGE

SEQ ID NO: 31 - protein, 61 aa, Artificial Sequence
  Signal peptide of Saccharomyces cerevisiae- derived mating pheromone N1-factor
MRFPSIFTAVLFAASSALAAPVNTTTEDETAQIPAEAVIGYLDLEGDFDVAVLPFSNSTNN

SEQ ID NO: 32 - protein, 67 aa, Artificial Sequence
  Signal peptide of Saccharomyces cerevisiae- derived chitin trans-glycosylase
MKVLDLLTVLSASSLLSTFAAAESTATADSTTAASSTASCNPLKTTGCTPDTALATSFSEDFSSSSK

SEQ ID NO: 33 - protein, 66 aa, Artificial Sequence
  Signal peptide of Saccharomyces cerevisiae- derived phospholipase B
MKLQSLLVSAAVLTSLTENVNAWSPNNSYVPANVTCDDDINLVREASGLSDNETEWLKKRDAYTKE

SEQ ID NO: 34 - protein, 100 aa, Artificial Sequence
  Signal peptide of Saccharomyces cerevisiae- derived cell wall protein related to glucanases
MKLSATTLTAASLIGYSTIVSALPYAADIDTGCTTTAHGSHQHKRAVAVTYVYETVTVDKNGQTVTPTSTEASSTVASTTTLISESSVTKSSSKVASSSE

SEQ ID NO: 35 - protein, 97 aa, Artificial Sequence
  Signal peptide of Saccharomyces cerevisiae- derived phospholipase B
MQLRNILQASSLISGLSLAADSSSTTGDGYAPSIIPCPSDDTSLVRNASGLSTAETDWLKKRDAYTKEALHSFLSRATSNFSDTSLLSTLFSSNSSN

SEQ ID NO: 36 - protein, 51 aa, Artificial Sequence
  Signal peptide of Saccharomyces cerevisiae- derived exo-1,3-N2 glucanase
MISPISFLSSLLCLTYLTSALPILPKREVVTRVHTASTTNVVTDFYSTTTE

SEQ ID NO: 37 - protein, 100 aa, Artificial Sequence
  Signal peptide of Saccharomyces cerevisiae- derived cell wall-associated protein involved in export of acetylated sterols
MLEFPISVLLGCLVAVKAQTTFPNFESDVLNEHNKFRALHVDTAPLTWSDTLATYAQNYADQYDCSGVLTHSDGPYGENLALGYTDTGAVDAWYGEISKY

SEQ ID NO: 38 - protein, 88 aa, Artificial Sequence
  Signal peptide of Saccharomyces cerevisiae- derived aspartic protease
MKLKTVRSAVLSSLFASQVLGKIIPAANKRDDDSNSKFVKLPFHKLYGDSLENVGSDKKPEVRLLKRADGYEEIIITNQQSFYSVDLE

SEQ ID NO: 39 - protein, 62 aa, Artificial Sequence
  Signal peptide of Saccharomyces cerevisiae- derived cell wall mannoprotein (1)
MVKLTSIAAGVAAIAATASATTTLAQSDERVNLVELGVYVSDIRAHLAQYYSFQVAHPTETY

SEQ ID NO: 40 - protein, 65 aa, Artificial Sequence
  Signal peptide of Saccharomyces cerevisiae- derived cell wall mannoprotein (2)
MVKLTSIVAGVAAIAAGVAAAPATTTLSPSDERVNLVELGVYVSDIRAHLAEYYMFQAAHPTETY

SEQ ID NO: 41 - protein, 38 aa, Artificial Sequence
  Signal peptide of Saccharomyces cerevisiae- derived mating pheromone N1-factor
MQPITTASTQATQKDKSSEKKDNYIIKGLFWDPACVIA

SEQ ID NO: 42 - protein, 53 aa, Artificial Sequence
  Signal peptide of Saccharomyces cerevisiae- derived sporulation-specific exo-1,3-b-glucanase
MVSFRGLTTLTLLFTKLVNCNPVSTKNRDSIQFIYKEKDSIYSAINNQAINEK

SEQ ID NO: 43 - protein, 18 aa, Artificial Sequence
  Signal peptide of Homo sapiens chymotrypsinogen
MAFLWLLSCWALLGTTFG

SEQ ID NO: 44 - protein, 14 aa, Artificial Sequence
  Signal peptide of Homo sapiens interleukin-2
MQLLSCIALILALV

SEQ ID NO: 45 - protein, 15 aa, Artificial Sequence
  Signal peptide of Homo sapiens trypsinogen-2
MNLLLILTFVAAAVA

SEQ ID NO: 46 - protein, 17 aa, Artificial Sequence
  Signal peptide of Metridia longa luciferase
MDIKVVFTLVFSALVQA

SEQ ID NO: 47 - protein, 16 aa, Artificial Sequence
  Signal peptide of Oikopleura dioica Oikosin 1
MLLLSALLLGLAHGYS

SEQ ID NO: 48 - protein, 18 aa, Artificial Sequence
  Signal peptide of Oikopleura dioica Oikosin 2A
MKLLASVLTIAAADYACC

SEQ ID NO: 49 - protein, 21 aa, Artificial Sequence
  Signal peptide of Oikopleura dioica Oikosin 3
MKISAGLLGVALGQNEGSAEA

SEQ ID NO: 50 - protein, 16 aa, Artificial Sequence
  Signal peptide of Oikopleura dioica Oikosin 4A
MKLFAALSAFSASVEA

SEQ ID NO: 51 - protein, 14 aa, Artificial Sequence
  Signal peptide of Oikopleura dioica Oikosin 5A
MKLLCSVLLGTVFG

SEQ ID NO: 52 - protein, 14 aa, Artificial Sequence
  Signal peptide of Oikopleura dioica Oikosin 6A
MKISPLLVVTAVVG

SEQ ID NO: 53 - protein, 18 aa, Artificial Sequence
  Signal peptide of Oikopleura dioica Oikosin 7A
MKIAATFAALASATEWQG

SEQ ID NO: 54 - protein, 18 aa, Artificial Sequence
  Signal peptide of Vargula hilgendorfii luciferase
MKIIILSVILAYCVTDNC

SEQ ID NO: 55 - protein, 28 aa, Artificial Sequence
  Signal peptide of Methanococcus jannaschii Slmj1
MAMSLKKIGAIAVGGAMVATALASGVAA

SEQ ID NO: 56 - protein, 22 aa, Artificial Sequence
  Signal peptide of Hepatitis C virus serotype 1b E1 protein
MGCSFSIFLLALLSCLTTPASA

SEQ ID NO: 57 - protein, 20 aa, Artificial Sequence
  Signal peptide of Hepatitis C virus serotype 1b E2 protein
MVGNWAKVLIVMLLFAGVDG

SEQ ID NO: 58 - protein, 25 aa, Artificial Sequence
  Signal peptide of tissue plasminogen activator
MDAMKRGLCCVLLLCGAVFVDSVTG

SEQ ID NO: 59 - protein, 19 aa, Artificial Sequence
  Artificial signal peptide
MDAMKVLLLVFVSPSQVTG

SEQ ID NO: 60 - protein, 12 aa, Artificial Sequence
  Epitope Frameshift of the HSD3B7 gene
KLWGLTPKVTPS

SEQ ID NO: 61 - protein, 9 aa, Artificial Sequence
  Epitope BCA(478-486)
ALWAWPSEL

SEQ ID NO: 62 - protein, 9 aa, Artificial Sequence
  Epitope CHI3L2(10-18)
SLWAGVVVL

SEQ ID NO: 63 - protein, 9 aa, Artificial Sequence
  Epitope CSPG4(21-29)
TMLARLASA

SEQ ID NO: 64 - protein, 9 aa, Artificial Sequence
  Epitope FABP7(118-126)
LTFGDVVAV

SEQ ID NO: 65 - protein, 9 aa, Artificial Sequence
  Epitope IGF2BP3(552-560)
KIQEILTQV

SEQ ID NO: 66 - protein, 9 aa, Artificial Sequence
  Epitope NLGN4X(131-139)
NLDTLMTYV

SEQ ID NO: 67 - protein, 9 aa, Artificial Sequence
  Epitope NRCAM(692-700)
GLWHHQTEV

SEQ ID NO: 68 - protein, 9 aa, Artificial Sequence
  Epitope PTPRZ1(195-203)
AIIDGVESV

SEQ ID NO: 69 - protein, 9 aa, Artificial Sequence
  Epitope PTPRZ1(1347-1355)
KVFAGIPTV

SEQ ID NO: 70 - protein, 9 aa, Artificial Sequence
  Epitope TNC(3-11)
AMTQLLAGV

SEQ ID NO: 71 - protein, 16 aa, Artificial Sequence
  Epitope gp100(44-59)
WNRQLYPEWTEAQRLD

SEQ ID NO: 72 - protein, 9 aa, Artificial Sequence
  Epitope gp100(209-217)
IMDQVPFSV

SEQ ID NO: 73 - protein, 9 aa, Artificial Sequence
  Epitope TRP-2(180-188)
SVYDFFVWL

SEQ ID NO: 74 - protein, 10 aa, Artificial Sequence
  Epitope from antigen IL-13RN12
LLDTNYNLFY

SEQ ID NO: 75 - protein, 9 aa, Artificial Sequence
  Epitope IL-13RN12(345-353)
ALPFGFILV

SEQ ID NO: 76 - protein, 9 aa, Artificial Sequence
  Epitope from antigen IL-13RN12 (2)
WLPFGFILI

SEQ ID NO: 77 - protein, 9 aa, Artificial Sequence
  Epitope from antigen MAGE A11
EVLSIMGVY

SEQ ID NO: 78 - protein, 8 aa, Artificial Sequence
  Epitope from antigen SSX5
SEKIIYVY

SEQ ID NO: 79 - protein, 8 aa, Artificial Sequence
  Epitope from antigen NOL4
INAVRQLV

SEQ ID NO: 80 - protein, 9 aa, Artificial Sequence
  Epitope from antigen MAGE C2
FVYGEPREL

SEQ ID NO: 81 - protein, 9 aa, Artificial Sequence
  Epitope EPHA2(883-891)
TLADFDPRV

SEQ ID NO: 82 - protein, 10 aa, Artificial Sequence
  Epitope YKL-40(201-210)
SIMTYDFHGA

SEQ ID NO: 83 - protein, 9 aa, Artificial Sequence
  Epitope from antigen VEGFR1
SYGVLLWEI

SEQ ID NO: 84 - protein, 9 aa, Artificial Sequence
  Epitope from antigen VEGFR2
RFVPDGNRI

SEQ ID NO: 85 - protein, 9 aa, Artificial Sequence
  Epitope SURVIVIN(96-104)
LMLGEFLKL

SEQ ID NO: 86 - protein, 9 aa, Artificial Sequence
  Epitope pp65(495-503)
NLVPMVATV

SEQ ID NO: 87 - protein, 9 aa, Artificial Sequence
  Epitope MART-1(27-35)
AAGIGILTV

SEQ ID NO: 88 - protein, 9 aa, Artificial Sequence
  Epitope HER2/NEU(369-377)
KIFGSLAFL

SEQ ID NO: 89 - protein, 9 aa, Artificial Sequence
  Epitope from antigen Tyrosinase
YMDGTMSQV

SEQ ID NO: 90 - protein, 9 aa, Artificial Sequence
  Epitope from antigen HB-EGF
KLLPSVVLK

SEQ ID NO: 91 - protein, 9 aa, Artificial Sequence
  Epitope from antigen EGFR (1)
STFDSPAHW

SEQ ID NO: 92 - protein, 10 aa, Artificial Sequence
  Epitope from antigen EGFR (2)
SPSTSRTPLL

SEQ ID NO: 93 - protein, 9 aa, Artificial Sequence
  Epitope from antigen PCNA
SHIGDAVVI

SEQ ID NO: 94 - protein, 9 aa, Artificial Sequence
  Epitope from antigen PTPRZ1 (1)
QHEGTVNIF

SEQ ID NO: 95 - protein, 10 aa, Artificial Sequence
  Epitope from antigen PTPRZ1 (2)
EVLDSHIHAY

SEQ ID NO: 96 - protein, 9 aa, Artificial Sequence
  Epitope from antigen ITGAV
FGVDRAILY

SEQ ID NO: 97 - protein, 9 aa, Artificial Sequence
  Epitope from antigen STAT-3
THSLPVVVI

SEQ ID NO: 98 - protein, 9 aa, Artificial Sequence
  Epitope from antigen IQGAP1
DHINDIIKI

SEQ ID NO: 99 - protein, 11 aa, Artificial Sequence
  Epitope from antigen HO-1 (1)
EVIPITPAMQR

SEQ ID NO: 100 - protein, 9 aa, Artificial Sequence
  Epitope from antigen HO-1 (2)
EVIPITPAM

SEQ ID NO: 101 - protein, 11 aa, Artificial Sequence
  Epitope from antigen BSG
EHINEGETAML

SEQ ID NO: 102 - protein, 9 aa, Artificial Sequence
  Epitope from antigen SEC61G
IHIPINNII

SEQ ID NO: 103 - protein, 9 aa, Artificial Sequence
  Epitope from antigen TNC
GLAPSIRTK

SEQ ID NO: 104 - protein, 9 aa, Artificial Sequence
  Epitope from antigen PIK3R1
SVVELINHY

SEQ ID NO: 105 - protein, 15 aa, Artificial Sequence
  Neoepitope from neoantigen PAPPA2
QVLLEGEHCWLGAKV

SEQ ID NO: 106 - protein, 14 aa, Artificial Sequence
  Neoepitope from neoantigen NF1
HRKSLLLTIFQWKF

SEQ ID NO: 107 - protein, 7 aa, Artificial Sequence
  Neoepitope from neoantigen ATP8B3
ILLSLGF

SEQ ID NO: 108 - protein, 15 aa, Artificial Sequence
  Neoepitope from neoantigen HOXA1
HHHHHHHRHPQPATY

SEQ ID NO: 109 - protein, 15 aa, Artificial Sequence
  Neoepitope from neoantigen OR4C3
RVLFVVFIYVVTVCG

SEQ ID NO: 110 - protein, 15 aa, Artificial Sequence
  Neoepitope from neoantigen FAM20B
LVVGRFVSLRTEIKP

SEQ ID NO: 111 - protein, 15 aa, Artificial Sequence
  Neoepitope from neoantigen INSM2
APLSAALKSLKRAAG

SEQ ID NO: 112 - protein, 15 aa, Artificial Sequence
  Neoepitope from neoantigen GOLGA6L22
IREQEEMTQEQEEKM

SEQ ID NO: 113 - protein, 15 aa, Artificial Sequence
  Neoepitope from neoantigen TMEM241
PTGDLFSILDFPFLY

SEQ ID NO: 114 - protein, 15 aa, Artificial Sequence
  Neoepitope from neoantigen POTEJ
DIKEKLCFVALDFEQ

SEQ ID NO: 115 - protein, 8 aa, Artificial Sequence
  Neoepitope from neoantigen PRKRA
MSQSRHRL

SEQ ID NO: 116 - protein, 15 aa, Artificial Sequence
  Neoepitope from neoantigen C9orf57
VILFRLLVVILFGRL

SEQ ID NO: 117 - protein, 15 aa, Artificial Sequence
  Neoepitope from neoantigen LILRB3
SNPHLLSLPSEPLEL

SEQ ID NO: 118 - protein, 15 aa, Artificial Sequence
  Neoepitope from neoantigen MYLK
WSIGVICSILVSGLS

SEQ ID NO: 119 - protein, 15 aa, Artificial Sequence
  Neoepitope from neoantigen ABCA2
TLVKRPAKPGGPQEP

SEQ ID NO: 120 - protein, 15 aa, Artificial Sequence
  Neoepitope from neoantigen ATP1A2
PDAVGKCGSAGIKVI

SEQ ID NO: 121 - protein, 15 aa, Artificial Sequence
  Neoepitope from neoantigen LINC00273 (1)
EGGPAAPHLGSRTAP

SEQ ID NO: 122 - protein, 15 aa, Artificial Sequence
  Neoepitope from neoantigen LINC00273 (2)
NRPTSGPRQRHTRRS

SEQ ID NO: 123 - protein, 15 aa, Artificial Sequence
  Neoepitope from neoantigen LINC00273 (3)
ADPIPSGRSPGPCGA

SEQ ID NO: 124 - protein, 12 aa, Artificial Sequence
  Neoepitope from neoantigen CDH7
DMYGTGQQSLYS

SEQ ID NO: 125 - protein, 15 aa, Artificial Sequence
  Neoepitope from neoantigen ELL
QSYKNDFSAEYSEYR

SEQ ID NO: 126 - protein, 15 aa, Artificial Sequence
  Neoepitope from neoantigen NCAN
ARKAKYNLHATVRYQ

SEQ ID NO: 127 - protein, 15 aa, Artificial Sequence
  Neoepitope from neoantigen TTN (1)
FARKLKDIHETLGFP

SEQ ID NO: 128 - protein, 15 aa, Artificial Sequence
  Neoepitope from neoantigen TTN (2)
EPDNIKYMISEEKGS

SEQ ID NO: 129 - protein, 15 aa, Artificial Sequence
  Neoepitope from neoantigen GPR50
AATSHPKHIKPATSH

SEQ ID NO: 130 - protein, 15 aa, Artificial Sequence
  Neoepitope from neoantigen LCE1F
SSGGCCGSSSGGCCS

SEQ ID NO: 131 - protein, 15 aa, Artificial Sequence
  Neoepitope from neoantigen GOLGA6Ll
REDAGAGEEDVGAGG

SEQ ID NO: 132 - protein, 15 aa, Artificial Sequence
  Neoepitope from neoantigen GOLGA6L2
IREQEEMIREQEAQR

SEQ ID NO: 133 - protein, 15 aa, Artificial Sequence
  Neoepitope from neoantigen LOC645752
PPTWSGRHAPGDRDN

SEQ ID NO: 134 - protein, 15 aa, Artificial Sequence
  Neoepitope from neoantigen DSPP
QFLIPTSFSVSSNSV

SEQ ID NO: 135 - protein, 15 aa, Artificial Sequence
  Neoepitope from neoantigen CRHBP
HPLPSAEQYIDFCES

SEQ ID NO: 136 - protein, 15 aa, Artificial Sequence
  Neoepitope from neoantigen TENM3
GKGVMLAISQGRVQT

SEQ ID NO: 137 - DNA, 1632 bp, Artificial Sequence
  Nucleic acid sequence of VSV-G/gp100(44- 59)/TRP2(180-188)
atgaagtgcctgctgtacctggccttcctgttcatcggcgtgaactgcaagtggaacagacagctgtaccccgagtggaccgaggcccagagactggataagcttaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagtagcgtgtacgacttcttcgtgtggctggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 138 - protein, 543 aa, Artificial Sequence
  Amino acid sequence of VSV-G/gp100(44- 59)/TRP2(180-188)
MKCLLYLAFLFIGVNCKWNRQLYPEWTEAQRLDKLKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSSVYDFFVWLEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 139 - DNA, 19 bp, Artificial Sequence
  Pirmer TRP2 forward
ccaggatgaccgtgagcaa

SEQ ID NO: 140 - DNA, 19 bp, Artificial Sequence
  Primer TRP2 reverse
gggcagtcagggaatggat

SEQ ID NO: 141 - DNA, 20 bp, Artificial Sequence
  Primer murine gp100 forward
ggagcttccttcccgtgctt

SEQ ID NO: 142 - DNA, 20 bp, Artificial Sequence
  Primer murine gp100 reverse
gctcccattgatgatggtgt

SEQ ID NO: 143 - DNA, 20 bp, Artificial Sequence
  Primer human gp100 forward
ataggtgctttgctggctgt

SEQ ID NO: 144 - DNA, 20 bp, Artificial Sequence
  Primer human gp100 reverse
acctgcccatctggcaatac

SEQ ID NO: 145 - DNA, 20 bp, Artificial Sequence
  Primer VSV-G forward
aactggcacaacgacctgat

SEQ ID NO: 146 - DNA, 20 bp, Artificial Sequence
  Primer VSV-G reverse
gatgtacttggggccgtacc

SEQ ID NO: 147 - DNA, 20 bp, Artificial Sequence
  Primer b-actin forward
actcctatgtgggtgacgag

SEQ ID NO: 148 - DNA, 23 bp, Artificial Sequence
  Primer b-actin reverse
catcttttcacggttggccttag

SEQ ID NO: 149 - protein, 9 aa, Artificial Sequence
  Controle antigen P815
LPYLGWLVF

(19) United States
(12) Patent Application Publication
(10) Pub. No.: US 2022/0331417 A1
(43) Pub. Date: Oct. 20, 2022

(54) MODIFIED VESICULAR STOMATITIS VIRUS GLYCOPROTEIN AND USES THEREOF FOR THE TREATMENT OF BRAIN TUMORS

(71) Applicant: UNIVERSITÉ CATHOLIQUE DE LOUVAIN, Louvain la Neuve (BE)

(72) Inventors: Gaëlle VANDERMEULEN, Hoeilaart (BE); Alessandra LOPES, Bruxelles (BE); Chiara BASTIANCICH, Marseille (FR); Véronique PRÉAT, Kraainem (BE)

(73) Assignee: UNIVERSITÉ CATHOLIQUE DE LOUVAIN, Louvain la Neuve (BE)

(21) Appl. No.: 17/642,756

(22) PCT Filed: Sep. 24, 2020
(86) PCT No.: PCT/EP2020/076788

§ 371 Date: Mar. 14, 2022

(30) Foreign Application Priority Data
Sep. 24, 2019 (EP) 19199334.4

(51) Int. Cl. A61K 39/00; A61K 39/385; A61P 35/00

(52) U.S. Cl. CPC A61K 39/001156; A61K 39/001192; A61K 39/385; A61P 35/00; A61K 2039/53; A61K 2039/6075; A61K 2039/70; A61K 2039/80

(57) ABSTRACT

A vaccine for treating and/or preventing a brain tumor. More particularly, a modified vesicular stomatitis virus glycoprotein (VSV-G) including at least one tumor antigen, or a fragment thereof, for use in preventing and/or treating a brain tumor in an individual in need thereof, when administered before a surgery intended to remove all or part of the tumor, such as, a tumor resection. The inventors have shown that vaccination of individual with a brain tumor with a vaccine including a nucleic acid sequence encoding a modified VSV-G may be combined to a tumor resection in order to ameliorate the prognostic of the individuals.

### FIELD OF INVENTION

The present invention relates to the field of vaccination, in particular to the field of methods and related compositions for the preparation and administration of nucleic acid-based vaccines. More particularly, the invention relates to a modified vesicular stomatitis virus glycoprotein (VSV-G) comprising tumor antigens, to be administered before a surgery intended to remove all or part of the tumor, such as, a tumor resection, and its use for the therapeutic vaccination against brain tumors and brain metastasis.

### BACKGROUND OF INVENTION

Brain tumors comprise a diverse group of neoplasms that are often malignant and refractory to treatment. Primary brain tumors include glioblastoma, anaplastic astrocytoma, meningioma, and oligodendroglial tumors. The annual incidence of primary brain tumors is around 1/7,140. The overall prognosis and survival of patients with primary brain tumors remain poor, with an aggregate 5-year survival rate of 20%.

Up to 30% of people with primary cancers in parts of the body other than the brain will develop brain metastases. Metastatic brain tumors are the most common complications of systemic cancers and can typically occur with primary tumors such as in lung, bronchus, melanoma, kidney, breast, colon, rectum and ovary.

Among the brain tumors, glioblastoma is an aggressive type of cancer that can occur in the brain or spinal cord. Glioblastomas represent the most frequent brain tumors in adults, with an annual incidence of around 1/33,330. Glioblastoma forms from cells called astrocytes that support nerve cells.

Glioblastoma can occur at any age, but tends to occur more often in older adults, 70% of cases being observed in patients between 45 and 70 of age. It can cause worsening headaches, changes in mood or personality, trouble speaking, double or blurred vision, nausea, vomiting and seizures.

Glioblastoma, also known as glioblastoma multiforme, can be very difficult to treat and a cure is often not possible. Treatments often merely slow down its progression and reduce signs and symptoms.

Currently, glioblastoma treatment options include, surgery, radiation therapy, chemotherapy, tumor treating fields therapy, targeted drug therapy.

Surgery to remove the glioblastoma is, to date, the gold standard, as acknowledged by the National Brain tumor Society in the United States. However, since glioblastoma develops into the normal brain tissue, complete removal is not possible in many cases. Therefore, patients having undergone surgery often receive further additional treatments to target the remaining tumoral cells.

That is why, radiation therapy is usually recommended after surgery and may be combined with chemotherapy. For patient who cannot undergo surgery, radiation therapy and chemotherapy may be used as a primary treatment.

After surgery, the chemotherapy drug temozolomide is often associated during and after radiation therapy. However, this drug can cause short-term side effects.

In addition, tumor treating fields (TTF) therapy, which uses an electrical field to disrupt the tumor cells' ability to multiply, may be combined with chemotherapy and may be recommended after radiation therapy.

Finally, targeted drugs therapy, such as Bevacizumab may be used to specifically target vascular endothelial growth factors to inhibit the formation of new blood vessels able to deliver blood and nutrients to cancer cells. Bevacizumab may be an option when glioblastoma recurs or does not respond to other treatments.

In general, the prognosis of primary and metastatic brain tumors, and in particular glioblastoma, is poor, in particular in the absence of total resection, in older patients and in case of severe neurological deficits.

In the last decades, a more targeted approach of cancer therapy is being under development, relying upon the use of nucleic acid vectors, in particular derived from viruses. The overall strategy is to boost the body's natural defenses to fight a cancer, in particular elicit an immune response within the body to specifically destroy the cancer cells.

As an example, WO2018/050738 relates to vaccines and methods for the treatment of a disease or condition, in particular a cancer or an infectious disease, based upon modified vesicular stomatitis virus glycoprotein (VSV-G).

However, in many cases vaccination by itself is not sufficient to elicit a proper immunological response so as to destroy the cancer cells.

Therefore, there is a need to provide the state of the art with new strategic approaches to prevent and/or treat brain tumors. There is also a need to provide the state of the art with new therapeutic approaches to ameliorate the prognostic, in particular the overall survival, of individuals with brain tumor.

### SUMMARY

This invention thus relates to a modified vesicular stomatitis virus glycoprotein (VSV-G) comprising at least one tumor antigen, or a fragment thereof, for use in preventing and/or treating a brain tumor in an individual in need thereof, wherein said modified VSV-G is to be administered before a surgery in said individual as to remove all or part of the tumor, in particular a tumor resection.

In certain embodiments, said modified VSV-G is further to be administered after a surgery in said individual as to remove all or part of the tumor, in particular a tumor resection. In some embodiments, said at least one tumor antigen is selected in (i) a group of antigens comprising ALK, GALT3, NA17-A, HSD3B7, BCAN, CHI3L2, CSPG4, FABP7, IGF2BP3, NLGN4X (Neuroligin 4, X-linked), NRCAM, PTPRZ1, TNC, AIM2, gp100, MAGE, TRP2, HER2, IL13Rα2, MAGE A11, SSX5, NOL4, MAGE C2, EphA2, YKL-40, VEGFR1, VEGFR2, SURVIVIN, pp65, IE1, MART-1, SART-1, HER2/NEU, GNT-V, Tyrosinase, hTERT, B-CYCLIN, IDH1, EGFRvIII, WT-1, HSPPC-96, HB-EGF, EGFR, PCNA, ITGAV, STAT-3, IQGAP-1, HO-1, BSG, SEC61G and PIK3R1, preferably selected in a group comprising gp100, TRP2, pp65 and EGFRvIII, or (ii) a group of neoantigens comprising PAPPA2, NF1, ATP8B3, HOXA1, OR4C3, FAM20B, INSM2, GOLGA6L22, TMEM241, POTEJ, PRKRA, C9orf57, LILRB3, MYLK, ABCA2, ATP1A2, LINC00273, CDH7, ELL, NCAN, TTN, GPR50, LCE1F, GOLGA6L1, GOLGA6L2, LOC645752, DSPP, CRHBP and TENM3. In certain embodiments, said at least one tumor antigen is gp100 and/or TRP2. In some embodiments, said at least one tumor antigen comprises an epitope selected in the group of epitopes of sequences SEQ ID NO: 60 to SEQ ID NO: 104 and of neoepitopes of sequences SEQ ID NO: 105 to SEQ ID NO: 136. In some embodiments, said at least one tumor antigen is inserted in a VSV-G comprising SEQ ID NO: 1. In certain embodiments, said at least one epitope is epitope gp100₄₄₋₅₉ of sequence SEQ ID NO: 71 and/or epitope TRP2180188 of sequence SEQ ID NO: 73. In some embodiments, epitope gp100₄₄₋₅₉ of sequence SEQ ID NO: 71 is inserted at VSV-G amino acid positions 18 of SEQ ID NO: 1 and/or epitope TRP2₁₈₀₋₁₈₈ of sequence SEQ ID NO: 73 is inserted at VSV-G amino acid positions 191 of SEQ ID NO: 1.

The invention also relates to a nucleic acid sequence encoding a modified vesicular stomatitis virus glycoprotein (VSV-G) according to the instant invention for use in preventing and/or treating a brain tumor in an individual in need thereof, wherein said nucleic acid sequence is to be administered before a surgery in said individual as to remove all or part of the tumor, in particular a tumor resection.

The invention further relates to a vector containing a nucleic acid sequence encoding a modified vesicular stomatitis virus glycoprotein VSV-G according to the invention for use in preventing and/or treating a brain tumor in an individual in need thereof, wherein said vector is to be administered before a surgery in said individual as to remove all or part of the tumor, in particular a tumor resection.

Another aspect of the invention relates to a dendritic cell population transfected by the nucleic acid sequence encoding the modified VSV-G or a vector according to the invention for use in preventing and/or treating a brain tumor in an individual in need thereof, wherein said dendritic cell population is to be administered before a surgery in said individual as to remove all or part of the tumor, in particular a tumor resection.

A still further aspect of the invention relates to a composition comprising a modified VSV-G, a nucleic acid sequence, a vector, or a dendritic cell population according to the invention for use in preventing and/or treating a brain tumor in an individual in need thereof, wherein said composition is to be administered before a surgery in said individual as to remove all or part of the tumor, in particular a tumor resection.

In another aspect, the invention relates to a vaccine comprising a modified VSV-G, a nucleic acid sequence, a vector, or a dendritic cell population according to the invention, and optionally at least one adjuvant, for use in preventing and/or treating a brain tumor in an individual in need thereof, wherein said vaccine composition is to be administered before a surgery in said individual as to remove all or part of the tumor, in particular a tumor resection. In some embodiments, said vaccine is a nucleic acid vaccine or a protein vaccine.

In some embodiments, said modified VSV-G, nucleic acid sequence, vector, dendritic cell population, composition, or vaccine is to be administered to the individual intramuscular injection, intradermal injection, intra-tumoral injection, peritumoral injection, gene gun, electroporation or sonoporation.

In some embodiments, the brain tumor is selected in the group consisting of glioblastoma, anaplastic astrocytoma, meningioma, and oligodendroglial tumor. In certain embodiments, the brain tumor is a glioblastoma.

A further aspect of the invention relates to a method for treating a brain tumor in an individual in need thereof, said method comprising the step of:
a) administering to said individual a therapeutically effective amount of the modified VSV-G, the nucleic acid sequence, the vector, the dendritic cell population, the composition, or the vaccine according to the present disclosure;
b) performing a surgery in said individual as to remove all or part of the tumor, in particular a brain tumor resection.

In another aspect, the invention pertains to method for ameliorating the prognostic of an individual with a brain tumor, said method comprising the steps of:
a) administering to said individual a therapeutically effective amount of the modified VSV-G, the nucleic acid sequence, the vector, the dendritic cell population, the composition, or the vaccine according to the instant disclosure; and
b) performing a surgery in said individual as to remove all or part of the tumor, in particular a brain tumor resection.

### Definitions

In the present invention, the following terms have the following meanings:
“Brain tumor” is intended to refer to a tumor (cancer) that is localized in the brain and/or the spinal cord. As used herein, the expression “brain tumor” encompasses both primary tumors localized in the brain and/or spinal cord, and metastatic brain tumors, resulting from metastasis of a primary tumor localized elsewhere in the body than the brain and/or the spinal cord. In some embodiments, the expressions “brain tumor” and “brain cancer” are intended to be equivalent and may be substitute one another.
“VSV-G” is intended to refer to the glycoprotein G of the vesicular stomatitis virus.
“Antigen” refers to any molecule that can initiate a cellular and/or humoral immune response in an individual, leading to the stimulation of B and/or T lymphocytes (also referred to as B cells and T cells, respectively). In some embodiments, an antigen is capable of being bound by an antibody or T cell receptor. The structural aspect of an antigen, e.g., three-dimensional conformation or modification (such as, e.g., phosphorylation), that gives rise to a biological response, is referred to herein as “epitope”, “antigenic determinant” or “antigen epitopic fragment”. By extension, a “tumor antigen” is intended to refer to a protein, a glycoprotein, a glycolipid, or a carbohydrate that is expressed by a tumor cell, in particular at its surface, and that is capable of eliciting an immune response in the host.
“Neoantigen” or “neoantigenic” refers to a class of tumor antigens that arises from one or several tumor-specific mutation(s) which alter(s) the amino acid sequence of genome encoded proteins.
The terms “epitope”, “antigenic determinant” and “antigen epitopic fragment” are intended to be used interchangeably. They all refer to the part of an antigen that is recognized by the immune system, specifically by antibodies, B cells or T cells. Epitopes can be formed both from contiguous amino acids or, alternatively, from non-contiguous amino acids that are juxtaposed by tertiary folding of a protein (therefore referred to as “conformational epitope”). Epitopes formed from contiguous amino acids are typically retained on exposure to denaturing solvents, whereas conformational epitopes are typically lost on treatment with denaturing solvents. They define the minimum binding site for an antibody, B cell or T cell, and thus represent the target of specificity of an antibody, B cell or T cell.
“T-cell epitope” refers to an epitope that can be bound by MHC molecules of class I or II in the form of a peptide-presenting MHC molecule or MHC complex and then, in this form, be recognized and bound by naïve T cells, cytotoxic CD8 T cells or T helper CD4 cells. T cell epitopes may be presented by MHC class I for CD8 T cell recognition (therefore referred to as CD8 T cell epitopes), by MHC class II for CD4 T cell recognition (therefore referred to as CD4 T cell epitopes or helper T cell epitopes), or by both.
“Peptide” refers to a linear polymer of amino acids of less than 50 amino acids linked together by peptide bonds; a “polypeptide” refers to a linear polymer of at least 50 amino acids linked together by peptide bonds; and a “protein” specifically refers to a functional entity formed of one or more peptides or polypeptides, and optionally of non-polypeptides cofactors.
“Signal peptide”, further referred to as “signal sequence”, “targeting signal”, “localization signal”, “localization sequence”, “transit peptide”, “leader sequence” or “leader peptide”, refers to a peptide, present at the N-terminus or at the C-terminus of a protein, used to address it to a particular cellular compartment, such as the nucleus, the endoplasmic reticulum, the Golgi, and the like. In one embodiment, the signal peptide of the invention comprises from 4 to 35 amino acids.
“Immunogenic composition” is intended to refer to a composition comprising an antigenic molecule, which is capable of eliciting, upon administration to an individual, a humoral and/or a cellular immune response in said individual. In some embodiments, the immunogenic composition may be introduced directly into a recipient individual, e.g., by injection, inhalation, oral, intranasal and mucosal administration.
“Vaccine” is intended to refer to any preparation comprising a substance or a group of substances meant to stimulate the immune system of an individual in order to promote a defensive response against a pathogen, such as a bacterium or a virus, or against a tumor. Prophylactic vaccines are used to prevent an individual from developing a particular disease or, at least, to develop an attenuated state of the disease. Such prophylactic vaccines usually comprise the pathogen responsible for the disease, either live and weakened (attenuated) or killed, or components thereof, such as, e.g., purified or recombinant components thereof. Therapeutic vaccines are intended to treat specific diseases in an individual, in particular cancers. Such therapeutic anti-cancer vaccines comprise a tumor-antigen or tumor-antigens, which are capable of eliciting an immune response directed against the tumor cells.
“Adjuvant” refers to a molecule that stimulates the immune response against an antigen and/or that modulates the immune response so as to obtain the expected response. In particular, the addition of adjuvants in vaccine formulations aims to improve, accelerate, shift and/or extend the specific immune response directed against the antigen(s) comprised in the vaccine formulations. The advantages of adjuvants include enhancing the immunogenicity of antigens, changing the nature of the immune response, reducing the amount of antigen(s) required to induce an effective immunization, reducing the frequency of booster immunizations, and enhancing the immune response in the elderly and the immunocompromised or immunodeficient individuals.
“Genetic adjuvant” refers to any biologically active factor, such as a cytokine, an interleukin, a chemokine, a ligand, and optimally combinations thereof, which is expressed by a vector, and which, when administered with a DNA vaccine encoding an antigen, enhances the antigen-specific immune response. Desirable genetic adjuvants include, but are not limited to, DNA sequences encoding: GM-CSF, interferons (IFNs) (e.g., IFN-α, IFN-β and IFN-γ), interleukins (ILs) (e.g., IL-1β, IL-2, IL-10, IL-12, IL-13), TNF-α, and combinations thereof. The genetic adjuvants may also be immunostimulatory polypeptide from Parapox virus, such as a polypeptide of Parapox virus strain D1701 or NZ2 or Parapox immunostimulatory polypeptides B2WL or PP30. Still other such biologically active factors that enhance the antigen-specific immune response may be readily selected by one of skill in the art, and a suitable plasmid vector containing the same factors constructed by known techniques (for a review on genetic adjuvant for DNA vaccines, see Calarota & Weiner, 2004. Expert Rev. Vaccines. 3: S135-49; Calarota & Weiner, 2004. Immunol. Rev. 199:84-99; Kutzler & Weiner, 2004. J. Clin. Invest. 14(9):1241-4).
In some embodiments, the genetic adjuvant is not encoded by a nucleic acid or a vector encoding a modified VSV-G according to the invention. In some embodiments, the genetic adjuvant is encoded by a nucleic acid or a vector encoding a modified VSV-G according to the invention. According to this embodiment, the genetic adjuvant can be under the control of its own promoter; or the genetic adjuvant can be under the control of the same promoter as the modified VSV-G according to the invention, separated therefrom by an Internal Ribosome Entry Site (IRES).
“Dendritic cells” refers to antigen-presenting cells of the immune system which present cytoplasmic branched projections called dendrites at certain development stages. Dendritic cells have the particular function to trigger the adaptive immune response induced in response to an antigen.
“Individual” refers to an animal, preferably a mammal, more preferably a human.
In some embodiments, an individual may be a mammal. Mammals include, but are not limited to, all primates (human and non-human), cattle (including cows), horses, pigs, sheep, goats, dogs, cats, and any other mammal which is awaiting the receipt of, or is receiving medical care or was/is/will be the object of a medical procedure, or is monitored for the development of a disease.
In some embodiments, an individual may be a “patient”, i.e., a warm-blooded animal, more preferably a human, who/which is awaiting the receipt of, or is receiving medical care or was/is/will be the object of a medical procedure, or is monitored for the development of a disease. In some embodiments, the individual is an adult (e.g., an individual above the age of 18). In some embodiments, the individual is a child (e.g., an individual below the age of 18). In some embodiments, the individual is a male. In some embodiments, the individual is a female.
“Treating” or “treatment” or “alleviation” refers to both therapeutic treatment and prophylactic or preventative measures; wherein the object is to prevent or slow down (lessen) the targeted pathologic condition or disorder, such as e.g., a cancer or an infection. Those in need of treatment include those already with the disorder as well as those prone to have the disorder or those in whom the disorder is to be prevented. An individual or mammal is successfully “treated” for a specific disease or condition, such as e.g., a cancer or an infection if, after receiving a therapeutic amount of a modified VSV-G, a nucleic acid, a vector, a dendritic cell population, a composition, or a vaccine according to the present invention, alone or in combination with another treatment, the patient shows observable and/or measurable reduction in or absence of one or more of the following: reduction in the number of pathogenic cells; reduction in the percent of total cells that are pathogenic; and/or relief to some extent, one or more of the symptoms associated with the specific disease or condition; reduced morbidity and mortality, and improvement in quality of life issues. The above parameters for assessing successful treatment and improvement in the disease are readily measurable by routine procedures familiar to a physician.
“Pharmaceutically acceptable excipient” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, for example, FDA Office or EMA.
“Therapeutically effective amount” is intended to refer to the level or amount of agent that is aimed at, without causing significant negative or adverse side effects to the target, (1) delaying or preventing the onset of a brain tumor; (2) slowing down or stopping the progression, aggravation, or deterioration of one or more symptoms of a brain tumor; (3) bringing about ameliorations of the symptoms of a brain tumor; (4) reducing the severity or incidence of a brain tumor; or (5) curing a brain tumor. A therapeutically effective amount may be administered prior to the onset of a brain tumor, for a prophylactic or preventive action. Alternatively, or additionally, the therapeutically effective amount may be administered after initiation of a brain tumor, for a therapeutic action.
“About” preceding a value means plus or less 10% of said value.

### DETAILED DESCRIPTION

The inventors have shown that vaccination of mice with brain tumor, namely a glioblastoma, with a plasmid encoding a modified VSV-G protein comprising inserted defined T cell epitopes (originating from gp100 and TRP2), was surprisingly able to generate a specific and long-lasting immune response against glioblastoma and to target residual glioblastoma cells in mice that had subsequently undergone surgical resection.

The inventors have experimentally shown in the examples below that the combined sequential treatment of vaccination and tumor resection is therapeutically efficient, and has been proven to be surprisingly synergistic when the survival of the individuals with brain tumors are at stake.

This invention relates to a modified vesicular stomatitis virus glycoprotein (VSV-G) comprising at least one tumor antigen, or a fragment thereof, for use in preventing and/or treating a brain tumor in an individual in need thereof.

The invention also relates to a modified vesicular stomatitis virus glycoprotein (VSV-G) comprising at least one tumor antigen, or a fragment thereof, for use in preventing and/or treating a brain tumor.

In some embodiments, said modified VSV-G is to be administered before a surgery in said individual as to remove all or part of the tumor, in particular a tumor resection.

Within the scope of the invention, the expression “at least one” includes 1, 2, 3, 4, 5, 6, 7, 8, 9, 10, 15, 20, 25, 30, 35, 40, 45, 50 or more.

Within the meaning of the present invention, the term “modified VSV-G” amounts to the equivalent terms “recombinant VSV-G”, “engineered VSV-G”, “chimeric VSV-G” and “mutant VSV-G”. All terms are used interchangeably throughout the present specification. In some embodiments, a chimeric VSV-G is a VSV-G comprising at least one tumor antigen. In some embodiments, a mutant VSV-G is an insertion mutant, wherein at least one tumor antigen is inserted into VSV-G. In some embodiments, the terms “modified”, “recombinant”, “engineered”, “chimeric” and “mutant” are applied in reference to a VSV-G wild-type protein.

In some embodiments, the nucleic acid encoding a modified VSV-G of the invention is an isolated nucleic acid. In some embodiments, the modified VSV-G of the invention is a recombinant modified VSV-G. In some embodiments, the modified VSV-G of the invention is an isolated modified VSV-G.

Vesicular stomatitis viruses are constitutive members of the genus Vesiculovirus of the family Rhabdoviridae. Their genome accounts for a single molecule of negative-sense RNA, that encodes five major proteins: glycoprotein (G), polymerase or large protein (L), phosphoprotein (P), matrix protein (M) and nucleoprotein (N). The glycoprotein of the vesicular stomatitis virus (VSV-G) is a transmembrane protein that functions as the surface coat of the wild-type viral particles.

The VSV-G protein presents a N-terminal ectodomain, a transmembrane region and a C-terminal cytoplasmic tail. It is exported to the cell surface via the trans Golgi network (endoplasmic reticulum and Golgi apparatus).

Presently, nine vesicular stomatitis virus (VSV) strains have been classified in the Vesiculovirus genus: vesicular stomatitis Indiana virus (VSIV), vesicular stomatitis Alagoas virus (VSAV), Carajás virus (CJSV), Chandipura virus (CHPV), Cocal virus (COCV), Isfahan virus (ISFV), Maraba virus (MARAV), vesicular stomatitis New Jersey virus (VSNJV) and Piry virus (PIRYV). Additionally, other stains have been provisionally classified in the Vesiculovirus genus: Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURV), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viraemia of carp virus (SVCV), Tupaia virus (TUPV), Ulcerative disease rhabdovirus (UDRV) and Yug Bogdanovac virus (YBV).

Among these viral strains, the nucleic acid encoding VSV-G show sequence similarities, as expressed by percentage of sequence identity.

Sequences alignments using MUSCLE (Multiple Sequence Comparison by Log-Expectation) are shown in Table 1 below.

The term “identity” or “identical”, when used in a relationship between the sequences of two or more polypeptides or nucleic acids, refers to the degree of sequence relatedness between polypeptides or nucleic acids, as determined by the number of matches between strings of two or more amino acid residues or nucleotides. “Identity” measures the percent of identical matches between the smaller of two or more sequences with gap alignments (if any) addressed by a particular mathematical model or computer program (i.e., “algorithms”) Identity of related polypeptides or nucleic acids can be readily calculated by known methods. Such methods include, but are not limited to, those described in Arthur M. Lesk, Computational Molecular Biology: Sources and Methods for Sequence Analysis (New-York: Oxford University Press, 1988); Douglas W. Smith, Biocomputing: Informatics and Genome Projects (New-York: Academic Press, 1993); Hugh G. Griffin and Annette M. Griffin, Computer Analysis of Sequence Data, Part 1 (New Jersey: Humana Press, 1994); Gunnar von Heinje, Sequence Analysis in Molecular Biology: Treasure Trove or Trivial Pursuit (Academic Press, 1987); Michael Gribskov and John Devereux, Sequence Analysis Primer (New York: M. Stockton Press, 1991); and Carillo et al., 1988. SIAM J. Appl. Math. 48(5):1073-1082. Preferred methods for determining identity are designed to give the largest match between the sequences tested. Methods of determining identity are described in publicly available computer programs. Preferred computer program methods for determining identity between two sequences include the GCG program package, including GAP (Devereux et al., 1984. Nucl. Acid. Res. 12(1 Pt 1):387-395; Genetics Computer Group, University of Wisconsin Biotechnology Center, Madison, Wis.), BLASTP, BLASTN, TBLASTN and FASTA (Altschul et al., 1990. J. Mol. Biol. 215(3):403-410). The BLASTX program is publicly available from the National Center for Biotechnology Information (NCBI) and other sources (BLAST Manual, Altschul et al. NCB/NLM/NIH Bethesda, Md. 20894; Altschul et al., 1990. J. Mol. Biol. 215(3):403-410). The well-known Smith Waterman algorithm may also be used to determine identity.

In practice, the nucleic acid identity percentage may be determined using the CLUSTAL W software (version 1.83) the parameters being set as follows:
for slow/accurate alignments: (1) Gap Open Penalty: 15; (2) Gap Extension Penalty: 6.66; (3) Weight matrix: IUB;
for fast/approximate alignments: (4) K-tuple (word) size: 2; (5) Gap Penalty: 5;

(6) No. of top diagonals: 5; (7) Window size: 4; (8) Scoring Method: PERCENT.

In practice, the amino acid identity percentage may also be determined using the CLUSTAL W software (version 1.83) the parameters being set as follows:
for slow/accurate alignments: (1) Gap Open Penalty: 10.00; (2) Gap Extension Penalty:0.1; (3) Protein weight matrix: BLOSUM;
for fast/approximate alignments: (5) Gap penalty: 3; (6) K-tuple (word) size: 1; (7) No. of top diagonals: 5; (8) Window size: 5; (9) Scoring Method: PERCENT.

In some embodiments, the vesicular stomatitis virus glycoprotein (VSV-G) is selected in a group comprising VSV-G from VSIV (VSIV-G), from VSNJV (VSNJV-G), from CHPV (CHPV-G), from COCV (COCV-G), from PIRYV (PIRYV-G), from ISFV (ISFV-G), from SVCV (SVCV-G), from VSAV (VSAV-G), from CJSV (CJSV-G) and from MARAV (MARAV-G). In some embodiments, VSV-G comprises or consists of a sequence selected in a group comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 10.

In certain embodiments, VSV-G comprises or consists of the sequence SEQ ID NO: 1.

In some embodiments, VSV-G is a variant of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In some embodiments, a variant of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 is a polypeptide having a sequence identity of at least 30%, preferably of at least 35, 40, 45, 50, 55, 60, 65, 70, 75, 80, 85, 90, 95, 96, 97, 98, 99% or more with respectively SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.

In another embodiment, a variant of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 comprises conservative amino acid substitutions as compared to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10, respectively.

As used herein, the expression “conservative amino acid substitution” is defined herein as an amino acid exchange within one of the following five groups:
small aliphatic, nonpolar or slightly polar residues: Ala, Ser, Thr, Pro, Gly;
polar, negatively charged residues and their amides: Asp, Asn, Glu, Gln;
polar, positively charged residues: His, Arg, Lys;
large, aliphatic, nonpolar residues: Met, Leu, Ile, Val, Cys;
large, aromatic residues: Phe, Tyr, Trp.

As used herein, “amino acids” are represented by their full name, their three letter code or their one letter code as well known in the art Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

As used herein, “amino acids” include both natural and synthetic amino acids, and both D and L amino acids. “Standard amino acid” or “naturally occurring amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. For example, naphtlylalanine can be substituted for tryptophan to facilitate synthesis. Other synthetic amino acids that can be substituted include, but are not limited to, L-hydroxypropyl, L-3,4-dihydroxyphenylalanyl, α-amino acids such as L-α-hydroxylysyl and D-α-methylalanyl, L-α-methylalanyl, β-amino acids, and isoquinolyl.

As used herein, “amino acid” also encompasses chemically modified amino acids, including, but not limited to, salts, amino acid derivatives (such as amides), and substitutions. Amino acids contained within the polypeptides of the present invention, and particularly at the carboxy- or amino-terminus, can be modified by methylation, amidation, acetylation or substitution with other chemical groups which can change the polypeptide's circulating half-life without adversely affecting their activity. Additionally, a disulfide linkage may be present or absent in the polypeptides of the invention.

In another embodiment, a variant of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 is a polypeptide wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) from respectively SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 is/are absent, or substituted by any amino acid, or wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) is/are added.

In some embodiments of the invention, the modified VSV-G as described herein above may be modified by means well-known in the art, for instance by the addition of one or more functional group such as a phosphate, acetate, lipid or carbohydrate group, and/or by the addition of one or more protecting group.

Illustratively, the modified VSV-G can be modified by the addition of one or more functional groups such as phosphate, acetate, or various lipids and carbohydrates. The modified VSV-G of the invention may also exist as protein derivatives. The term “protein derivative” refers to compound having an amino group (—NH—), and more particularly, a peptide bond. Modified VSV-G may be regarded as substituted amides. Like the amide group, the peptide bond shows a high degree of resonance stabilization. The C—N single bond in the peptide linkage has typically about 40 percent double-bond character and the C═O double bond about 40 percent single-bond character. “Protecting groups” are those groups that prevent undesirable reactions (such as proteolysis) involving unprotected functional groups. Specific examples of amino protecting groups include formyl; trifluoroacetyl; benzyloxycarbonyl; substituted benzyloxycarbonyl such as (ortho- or para-) chlorobenzyloxycarbonyl and (ortho- or para-) bromobenzyloxycarbonyl; and aliphatic oxycarbonyl such as t-butoxycarbonyl and t-amiloxycarbonyl. The carboxyl groups of amino acids can be protected through conversion into ester groups. The ester groups include benzyl esters, substituted benzyl esters such as methoxybenzyl ester; alkyl esters such as cyclohexyl ester, cycloheptyl ester or t-butyl ester. The guanidino moiety may be protected by nitro; or arylsulfonyl such as tosyl, methoxybenzensulfonyl or mesitylenesulfonyl, even though it does not need a protecting group. The protecting groups of imidazole include tosyl, benzyl and dinitrophenyl. The indole group of tryptophan may be protected by formyl or may not be protected.

In some embodiments, the modified VSV-G of the invention comprises a signal peptide at the N-terminus of said modified VSV-G. In some embodiments, the modified VSV-G of the invention comprises a signal peptide at the C-terminus of said modified VSV-G.

In certain embodiments, the signal peptide comprises or consists of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acid residues.

In one embodiment, the signal peptide of the modified VSV-G of the invention comprises or consists of SEQ ID NO: 11 (MKCLLYLAFLFIGVNC).

In another embodiment, the signal peptide of the modified VSV-G of the invention comprises or consists of the Gaussia princeps luciferase signal peptide with SEQ ID NO: 12 (MGVKVLFALICIAVAEA).

In another embodiment, the signal peptide of the modified VSV-G of the invention comprises of consists of any of the signal peptides disclosed in Kober et al., 2013. Biotechnol. Bioeng. 110:1164-1173; Mori et al., 2015. J. Biosci. Bioeng. 120(5):518-525; Stern et al., 2007. Trends Cell Mol. Bio. 2:1-17; Wen et al., 2011. Acta Biochim Biophys Sin. 43:96-102. These include, without limitation:
the signal peptide of the Mus musculus Ig kappa light chain precursor (mutant A2) comprising or consisting of SEQ ID NO: 13 (MDMRAPAGIFGFLLVLFPGYRS);
the signal peptide of the Homo sapiens serum albumin preproprotein comprising or consisting of SEQ ID NO: 14 (MKWVTFISLLFLFSSAYS);
the signal peptide of the Homo sapiens immunoglobulin heavy chain comprising or consisting of SEQ ID NO: 15 (MDWTWRVFCLLAVTPGAHP);
the signal peptide of the Homo sapiens immunoglobulin light chain comprising or consisting of SEQ ID NO: 16 (MAWSPLFLTLITHCAGSWA);
the signal peptide of the Homo sapiens azurocidin preproprotein comprising or consisting of SEQ ID NO: 17 (MTRLTVLALLAGLLASSRA);
the signal peptide of the Homo sapiens Cystatin-S precursor comprising or consisting of SEQ ID NO: 18 (MARPLCTLLLLMATLAGALA);
the signal peptide of the Pseudopleuronectes americanus trypsinogen 2 precursor comprising or consisting of SEQ ID NO: 19 (MRSLVFVLLIGAAFA);
the signal peptide of the Mesobuthus martensii potassium channel blocker comprising or consisting of SEQ ID NO: 20 (MSRLFVFILIALFLSAIIDVMS);
the signal peptide of the Conus leopardus α-conotoxin 1p1.3 comprising or consisting of SEQ ID NO: 21 (MGMRMMFIMFMLVVLATTVVS);
the signal peptide of the Saccharomyces cerevisiae α-galactosidase (mutant m3) comprising or consisting of SEQ ID NO: 22 (MRAFLFLTACISLPGVFG);
the signal peptide of the Aspergillus niger cellulase comprising or consisting of SEQ ID NO: 23 (MKFQSTLLLAAAAGSALA);
the signal peptide of the Nepenthes gracilis aspartic proteinase nepenthesin-1 comprising or consisting of SEQ ID NO: 24 (MASSLYSFLLALSIVYIFVAPTHS);
the signal peptide of the Nepenthes rafflesiana acid chitinase comprising or consisting of SEQ ID NO: 25 (MKTHYSSAILPILTLFVFLSINPSHG);
the signal peptide of the M28 virus K28 prepro-toxin comprising or consisting of SEQ ID NO: 26 (MESVSSLFNIFSTIMVNYKSLVLALLSVSNLKYARG);
the signal peptide of the Zygosaccharomyces bailii killer toxin zygocin precursor comprising or consisting of SEQ ID NO: 27 (MKAAQILTASIVSLLPIYTSA);
the signal peptide of the Vibrio cholerae 0139 cholera toxin comprising or consisting of SEQ ID NO: 28 (MIKLKFGVFFTVLLSSAYA);
the signal peptide of the Saccharomyces cerevisiae-derived adhesion subunit of α-agglutinin comprising or consisting of SEQ ID NO: 29 (MQLLRCFSIFSVIASVLAQELTTICEQIPSPTLESTPYSLSTTTILANGK);
the signal peptide of the Saccharomyces cerevisiae-derived exo-1,3-β glucanase comprising or consisting of SEQ ID NO: 30 (MLSLKTLLCTLLTVSSVLATPVPARDPSSIQFVHEENKKRYYDYDHGSLGE);
the signal peptide of the Saccharomyces cerevisiae-derived mating pheromone α-factor comprising or consisting of SEQ ID NO: 31 (MRFPSIFTAVLFAASSALAAPVNTTTEDETAQIPAEAVIGYLDLEGDFDVAV LPFSNSTNN);
the signal peptide of the Saccharomyces cerevisiae-derived chitin trans-glycosylase comprising or consisting of SEQ ID NO: 32 (MKVLDLLTVLSASSLLSTFAAAESTATADSTTAASSTASCNPLKTTGCTPDT ALATSFSEDFSSSSK);
the signal peptide of the Saccharomyces cerevisiae-derived phospholipase B comprising or consisting of SEQ ID NO: 33 (MKLQSLLVSAAVLTSLTENVNAWSPNNSYVPANVTCDDDINLVREASGLSD NETEWLKKRDAYTKE);
the signal peptide of the Saccharomyces cerevisiae-derived cell wall protein related to glucanases comprising or consisting of SEQ ID NO: 34 (MKLSATTLTAASLIGYSTIVSALPYAADIDTGCTTTAHGSHQHKRAVAVTY VYETVTVDKNGQTVTPTSTEASSTVASTTTLISESS VTKSS SKVASSSE);
the signal peptide of the Saccharomyces cerevisiae-derived phospholipase B comprising or consisting of SEQ ID NO: 35 (MQLRNILQASSLISGLSLAADSSSTTGDGYAPSIIPCPSDDTSLVRNASGLSTA ETDWLKKRDAYTKEALHSFLSRATSNFSDTSLLSTLFSSNSSN);
the signal peptide of the Saccharomyces cerevisiae-derived exo-1,3-β glucanase comprising or consisting of SEQ ID NO: 36 (MISPISFLSSLLCLTYLTSALPILPKREVVTRVHTASTTNVVTDFYSTTTE);
the signal peptide of the Saccharomyces cerevisiae-derived cell wall-associated protein involved in export of acetylated sterols comprising or consisting of SEQ ID NO: 37 (MLEFPISVLLGCLVAVKAQTTFPNFESDVLNEHNKFRALHV DTAPLTWSDTLATYAQNYADQYDCSGVLTHSDGPYGENLALGYTDTGAVD AWYGEISKY);
the signal peptide of the Saccharomyces cerevisiae-derived aspartic protease comprising or consisting of SEQ ID NO: 38 (MKLKTVRSAVLSSLFASQVLGKIIPAANKRDDDSNSKFVKLPFHKLYGDSLE NVGSDKKPEVRLLKRADGYEEIIITNQQSFYSVDLE);
the signal peptide of the Saccharomyces cerevisiae-derived cell wall mannoprotein comprising or consisting of SEQ ID NO: 39 (MVKLTSIAAGVAAIAATASATTTLAQSDERVNLVELGVYVSDIRAHLAQYY SFQVAHPTETY);
the signal peptide of the Saccharomyces cerevisiae-derived cell wall mannoprotein comprising or consisting of SEQ ID NO: 40 (MVKLTSIVAGVAAIAAGVAAAPATTTLSPSDERVNLVELGVYVSDIRAHLA EYYMFQAAHPTETY);
the signal peptide of the Saccharomyces cerevisiae-derived mating pheromone α-factor comprising or consisting of SEQ ID NO: 41 (MQPITTASTQATQKDKSSEKKDNYIIKGLFWDPACVIA);
the signal peptide of the Saccharomyces cerevisiae-derived sporulation-specific exo-1,3-b-glucanase comprising or consisting of SEQ ID NO: 42 (MVSFRGLTTLTLLFTKLVNCNPVSTKNRDSIQFIYKEKDSIYSAINNQAINEK);
the signal peptide of the Homo sapiens chymotrypsinogen comprising or consisting of SEQ ID NO: 43 (MAFLWLLSCWALLGTTFG);
the signal peptide of the Homo sapiens interleukin-2 comprising or consisting of SEQ ID NO: 44 (MQLLSCIALILALV);
the signal peptide of the Homo sapiens trypsinogen-2 comprising or consisting of SEQ ID NO: 45 (MNLLLILTFVAAAVA);
the signal peptide of the Metridia longa luciferase comprising or consisting of SEQ ID NO: 46 (MDIKVVFTLVFSALVQA);
the signal peptide of the Oikopleura dioica Oikosin 1 comprising or consisting of SEQ ID NO: 47 (MLLLSALLLGLAHGYS);
the signal peptide of the Oikopleura dioica Oikosin 2A comprising or consisting of SEQ ID NO: 48 (MKLLASVLTIAAADYACC);
the signal peptide of the Oikopleura dioica Oikosin 3 comprising or consisting of SEQ ID NO: 49 (MKISAGLLGVALGQNEGSAEA);
the signal peptide of the Oikopleura dioica Oikosin 4A comprising or consisting of SEQ ID NO: 50 (MKLFAALSAFSASVEA);
the signal peptide of the Oikopleura dioica Oikosin 5A comprising or consisting of SEQ ID NO: 51 (MKLLCSVLLGTVFG);
the signal peptide of the Oikopleura dioica Oikosin 6A comprising or consisting of SEQ ID NO: 52 (MKISPLLVVTAVVG);
the signal peptide of the Oikopleura dioica Oikosin 7A comprising or consisting of SEQ ID NO: 53 (MKIAATFAALASATEWQG);
the signal peptide of the Vargula hilgendorfii luciferase comprising or consisting of SEQ ID NO: 54 (MKIIILSVILAYCVTDNC);
the signal peptide of the Methanococcus jannaschii Slmj1 comprising or consisting of SEQ ID NO: 55 (MAMSLKKIGAIAVGGAMVATALASGVAA);
the signal peptide of the Hepatitis C virus serotype 1b E1 protein comprising or consisting of SEQ ID NO: 56 (MGCSFSIFLLALLSCLTTPASA);
the signal peptide of the Hepatitis C virus serotype 1b E2 protein comprising or consisting of SEQ ID NO: 57 (MVGNWAKVLIVMLLFAGVDG);
the signal peptide of the tissue plasminogen activator comprising or consisting of SEQ ID NO: 58 (MDAMKRGLCCVLLLCGAVFVDSVTG); and
the signal peptide comprising or consisting of SEQ ID NO: 59 (MDAMKVLLLVFVSPSQVTG).

In some embodiments, the tumor antigen is selected in a group comprising a tumor-specific antigen (TSA), a tumor-associated antigen (TAA) and a cancer-germline/cancer testis antigen (CTA).

In some embodiments, the tumor antigen is a glioblastoma antigen. In certain embodiments, a glioblastoma antigen suitable for implementing the present invention includes, but is not limited to ALK (Anaplastic Lymphoma Kinase), GALT3 (Beta-1,3-N-Acetylgalactosaminyltransferase 1), NA17-A, HSD3B7 (Hydroxy-Delta-5-Steroid Dehydrogenase, 3 Beta- And Steroid Delta-Isomerase 7), BCAN (Brevican), CHI3L2 (Chitinase 3-like 2), CSPG4 (Chondroitin sulphate proteoglycan 4), FABP7 (Fatty acid-binding protein 7, brain), IGF2BP3 (Insulin-like growth factor 2 messenger RNA-binding protein 3), NLGN4X (Neuroligin 4, X-linked), NRCAM (Neuronal cell adhesion molecule), PTPRZ1 (Protein tyrosine phosphatase, receptor-type, Z polypeptide 1), TNC (TENASCIN C), AIM2 (Absent In Melanoma 2), gp100, MAGE (Melanoma Antigen Family), TRP2, HER2 (Erb-B2 Receptor Tyrosine Kinase 2), IL13Rα2, MAGE A11, SSX5 (Synovial Sarcoma, X Breakpoint 5), NOL4 (Nucleolar Protein 4), MAGE C2, EPHA2 (Ephrin Type-A Receptor 2), YKL-40 (Chitinase 3 Like 1), VEGFR1 (Vascular Endothelial Growth Factor Receptor 1), VEGFR2 (Vascular Endothelial Growth Factor Receptor 2), SURVIVIN, PP65, IE1, MART-1 (Melanoma Antigen Recognized By T-Cells 1), SART-1 (Squamous Cell Carcinoma Antigen Recognized By T-Cells 1), HER2/NEU, GNT-V (beta1,6-N-acetylglucosaminyltransferase V), Tyrosinase, hTERT (Telomerase Reverse Transcriptase), B-CYCLIN, IDH1 (Isocitrate Dehydrogenase 1), EGFRvIII, WT-1 (Wilm's tumor protein-1), HSPPC-96, HB-EGF (Heparin-binding EGF-like growth factor), EGFR (Epidermal Growth Factor Receptor), PCNA (Proliferating Cell Nuclear Antigen), ITGAV (Integrin alpha V), STAT-3 (Signal Transducer and Activator of Transcription 3), IQGAP-1 (IQ motif containing GTPase activating protein 1), HO-1 (Heme Oxygenase 1), BSG (Basigin), SEC61G (SEC61 gamma subunit) and PIK3R1 (Phosphoinositide 3-kinase regulatory subunit 1).

In some embodiments, said at least one tumor antigen is selected in (i) a group of antigens comprising ALK, GALT3, NA17-A, HSD3B7, BCAN, CHI3L2, CSPG4, FABP7, IGF2BP3, NLGN4X (Neuroligin 4, X-linked), NRCAM, PTPRZ1, TNC, AIM2, gp100, MAGE, TRP2, HER2, IL13Rα2, MAGE A11, SSX5, NOL4, MAGE C2, EPHA2, YKL-40, VEGFR1, VEGFR2, SURVIVIN, pp65, IE1, MART-1, SART-1, HER2/NEU, GNT-V, Tyrosinase, hTERT, B-CYCLIN, IDH1, EGFRvIII, WT-1, HSPPC-96, HB-EGF, EGFR, PCNA, ITGAV, STAT-3, IQGAP-1, HO-1, BSG, SEC61G and PIK3R1, preferably selected in a group comprising gp100, TRP2, pp65 and EGFRvIII, or (ii) a group of neoantigens comprising PAPPA2, NF1, ATP8B3, HOXA1, OR4C3, FAM20B, INSM2, GOLGA6L22, TMEM241, POTEJ, PRKRA, C9orf57, LILRB3, MYLK, ABCA2, ATP1A2, LINC00273, CDH7, ELL, NCAN, TTN, GPR50, LCE1F, GOLGA6L1, GOLGA6L2, LOC645752, DSPP, CRHBP and TENM3.

In certain embodiments, said at least one tumor antigen is selected in a group comprising ALK, GALT3, NA17-A, HSD3B7, BCAN, CHI3L2, CSPG4, FABP7, IGF2BP3, NLGN4X (Neuroligin 4, X-linked), NRCAM, PTPRZ1, TNC, AIM2, gp100, MAGE, TRP2, HER2, IL13Rα2, MAGE A11, SSX5, NOL4, MAGE C2, EPHA2, YKL-40, VEGFR1, VEGFR2, SURVIVIN, pp65, IE1, MART-1, SART-1, HER2/NEU, GNT-V, Tyrosinase, hTERT, B-CYCLIN, IDH1, EGFRvIII, WT-1, HSPPC-96, HB-EGF, EGFR, PCNA, ITGAV, STAT-3, IQGAP-1, HO-1, BSG, SEC61G and PIK3R1, preferably selected in a group comprising gp100, TRP2, pp65 and EGFRvIII.

In some embodiments, the tumor antigens described above are of use to treat and/or prevent a primary brain tumor.

In certain embodiments, the at least one tumor antigen is gp100 and/or TRP2.

In some embodiments, the modified VSV-G according to the invention comprises gp100 and/or TRP2 tumor antigen(s), or a fragment thereof.

In some embodiments, the at least tumor antigen of the invention is an epitope derived from an antigen described hereinabove. Accordingly, in certain embodiments, a fragment of antigen of the invention comprises, consists essentially of, or consists of, an epitope or “antigen epitopic fragment”. In some embodiments, a fragment of antigen of the invention comprises, consists essentially of, or consists of, more than one, i.e., at least two, three, four, five or more epitopes or “antigen epitopic fragments”.

In some embodiments, the epitope may be any epitope known from the person skilled in the art.

Illustratively, glioblastoma epitopes are notably described in Saikali et al. (Journal of neuro-oncology. 2007; 81(2):139-48); Myers et al. (Cancer Immunology, Immunotherapy. 2011; 60(9):1319-32); Dutoit et al. (Brain: a journal of neurology. 2012; 135(Pt 4):1042-54); Cuoco et al. (World Neurosurgery. 2018; 120:302-15); Phuphanich et al. (Cancer Immunology, Immunotherapy. 2013; 62(1):125-35); Zhang et al. (Clinical cancer research: an official journal of the American Association for Cancer Research. 2007; 13(2 Pt 1):566-75); Okada et al. (Journal of clinical oncology: official journal of the American Society of Clinical Oncology. 2011; 29(3):330-6); Pollack et al. Neuro Oncol. 2016; 18(8):1157-68); Batich et al. (Clinical cancer research: an official journal of the American Association for Cancer Research. 2017; 23(8):1898-909); Swartz et al. (Expert Opin Biol Ther. 2018; 18(11):1159-70).

In addition, glioblastoma epitopes according to the invention are notably described in https://media.springernature.com/original/springer-static/image/art%3A10.1007/02Fs00 401-018-1836-9/MediaObjects/401_2018_1836_FIG. 5_HTML.gif; and further, in https://mediaspringernaturecom/original/springer-static/image/ar0/03A101007/02Fs004 01-018-1836-9/MediaObjects/401_2018_1836_FIG. 5_HTML.gif; Kikuchi et al. (J Clin Med. 2019; 8(2):263).

In some embodiments, the epitope is capable of inducing an immune response against tumor antigens. Accordingly, in certain embodiments, the epitope is a tumoral epitope, preferably, the epitope is a tumoral CD4 T cell epitope and/or a tumoral CD8 T cell epitope. In some embodiments, the tumoral T cell epitope is a tumoral T cell epitope presented by MHC class I molecules. In some embodiments, the tumoral T cell epitope is a tumoral T cell epitope presented by MHC class II molecules.

In certain embodiments, the tumoral epitope, in particular the glioblastoma epitope is selected in the group of epitopes of sequences SEQ ID NO: 60 to SEQ ID NO: 104, as depicted in Table 2.

TABLE 2
Examples of glioblastoma epitopes.

| SEQ | | |
|---|---|---|
| ID | Epitope | |
| NO: | sequence | Antigen/Description |
| 60 | KLWGLTPKVTPS | Frameshift of the HSD3B7 |
| | | gene |
| 61 | ALWAWPSEL | BCA₄₇₈₋₄₈₆ |
| 62 | SLWAGVVVL | CHI3L2₁₀₋₁₈ |
| 63 | TMLARLASA | CSPG4₂₁₋₂₉ |
| 64 | LTFGDVVAV | FABP7₁₁₈₋₁₂₆ |
| 65 | KIQEILTQV | IGF2BP3₅₅₂₋₅₆₀ |
| 66 | NLDTLMTYV | NLGN4X₁₃₁₋₁₃₉ |
| 67 | GLWHHQTEV | NRCAM₆₉₂₋₇₀₀ |
| 68 | AIIDGVESV | PTPRZ1₁₉₅₋₂₀₃ |
| 69 | KVFAGIPTV | PTPRZ1₁₃₄₇₋₁₃₅₅ |
| 70 | AMTQLLAGV | TNC₃₋₁₁ |
| 71 | WNRQLYPEWTEAQRLD | gp100₄₄₋₅₉ |
| 72 | IMDQVPFSV | gp100₂₀₉₋₂₁₇ |
| 73 | SVYDFFVWL | TRP-2₁₈₀₋₁₈₈ |
| 74 | LLDTNYNLFY | IL-13Rα2 |
| 75 | ALPFGFILV | IL-13Rα2₃₄₅₋₃₅₃ |
| 76 | WLPFGFILI | IL-13Rα2 |
| 77 | EVLSIMGVY | MAGE A11 |
| 78 | SEKIIYVY | SSX5 |
| 79 | INAVRQLV | NOL4 |
| 80 | FVYGEPREL | MAGE C2 |
| 81 | TLADFDPRV | EPHA2₈₈₃₋₈₉₁ |
| 82 | SIMTYDFHGA | YKL-40₂₀₁₋₂₁₀ |
| 83 | SYGVLLWEI | VEGFR1 |
| 84 | RFVPDGNRI | VEGFR2 |
| 85 | LMLGEFLKL | SURVIVIN₉₆₋₁₀₄ |
| 86 | NLVPMVATV | pp65₄₉₅₋₅₀₃ |
| 87 | AAGIGILTV | MART-1₂₇₋₃₅ |
| 88 | KIFGSLAFL | HER2/NEU₃₆₉₋₃₇₇ |
| 89 | YMDGTMSQV | Tyrosinase |
| 90 | KLLPSVVLK | HB-EGF |
| 91 | STFDSPAHW | EGFR |
| 92 | SPSTSRTPLL | EGFR |
| 93 | SHIGDAVVI | PCNA |
| 94 | QHEGTVNIF | PTPRZ 1 |
| 95 | EVLDSHIHAY | PTPRZ 1 |
| 96 | FGVDRAILY | ITGAV |
| 97 | THSLPVVVI | STAT-3 |
| 98 | DHINDIIKI | IQGAP 1 |
| 99 | EVIPITPAMQR | HO-1 |
| 100 | EVIPITPAM | HO-1 |
| 101 | EHINEGETAML | BSG |
| 102 | IHIPINNII | SEC61G |
| 103 | GLAPSIRTK | TNC |
| 104 | SVVELINHY | PIK3R1 |

In some embodiments, the modified VSV-G according to the invention comprises epitope(s) gp100₄₄₋₅₉ (SEQ ID NO: 71) and/or TRP-2₁₈₀₋₁₈₈ (SEQ ID NO: 73).

In some embodiments, the antigen of the invention is a neoantigen, in particular a glioblastoma neoantigen.

As used herein, the term “neoantigen” is a newly formed antigen that has not been previously recognized by the immune system. Neoantigens and, by extension, neoantigenic determinants (or neoepitopes), can be formed when a protein undergoes further modification within a biochemical pathway such as glycosylation, phosphorylation or proteolysis.

In certain embodiments, a glioblastoma neoantigen suitable for implementing the present invention may be described in Valentini et al. (Oncotarget. 2018 Apr. 13; 9(28): 19469-19480), and includes, but is not limited to PAPPA2, NF1, ATP8B3, HOXA1, OR4C3, FAM20B, INSM2, GOLGA6L22, TMEM241, POTEJ, PRKRA, C9orf57, LILRB3, MYLK, ABCA2, ATP1A2, LINC00273, CDH7, ELL, NCAN, TTN, GPR50, LCE1F, GOLGA6L1, GOLGA6L2, LOC645752, DSPP, CRHBP and TENM3.

In certain embodiments, the tumoral neoepitope, in particular the glioblastoma neoepitope is selected in the group of epitopes of sequences SEQ ID NO: 105 to SEQ ID NO: 136, as depicted in Table 3.

TABLE 3
Examples of glioblastoma neoepitopes.

| SEQ | | |
|---|---|---|
| ID | Neoepitope | |
| NO: | sequence | Neoantigen/Description |
| 105 | QVLLEGEHCWLGAKV | PAPPA2 |
| 106 | HRKSLLLTIFQWKF | NF1 |
| 107 | ILLSLGF | ATP8B3 |
| 108 | HHHHHHHRHPQPATY | HOXA1 |
| 109 | RVLFVVFIYVVTVCG | OR4C3 |
| 110 | LVVGRFVSLRTEIKP | FAM20B |
| 111 | APLSAALKSLKRAAG | INSM2 |
| 112 | IREQEEMTQEQEEKM | GOLGA6L22 |
| 113 | PTGDLFSILDFPFLY | TMEM241 |
| 114 | DIKEKLCFVALDFEQ | POTEJ |
| 115 | MSQSRHRL | PRKRA |
| 116 | VILFRLLVVILFGRL | C9orf57 |
| 117 | SNPHLLSLPSEPLEL | LILRB3 |
| 118 | WSIGVICSILVSGLS | MYLK |
| 119 | TLVKRPAKPGGPQEP | ABCA2 |
| 120 | PDAVGKCGSAGIKVI | ATP1A2 |
| 121 | EGGPAAPHLGSRTAP | LINC00273 |
| 122 | NRPTSGPRQRHTRRS | LINC00273 |
| 123 | ADPIPSGRSPGPCGA | LINC00273 |
| 124 | DMYGTGQQSLYS | CDH7 |
| 125 | QSYKNDFSAEYSEYR | ELL |
| 126 | ARKAKYNLHATVRYQ | NCAN |
| 127 | FARKLKDIHETLGFP | TTN |
| 128 | EPDNIKYMISEEKGS | TTN |
| 129 | AATSHPKHIKPATSH | GPR50 |
| 130 | SSGGCCGSSSGGCCS | LCE1F |
| 131 | REDAGAGEEDVGAGG | GOLGA6L1 |
| 132 | IREQEEMIREQEAQR | GOLGA6L2 |
| 133 | PPTWSGRHAPGDRDN | LOC645752 |
| 134 | QFLIPTSFSVSSNSV | DSPP |
| 135 | HPLPSAEQYIDFCES | CRHBP |
| 136 | GKGVMLAISQGRVQT | TENM3 |

In some embodiments, said at least one tumor antigen comprises an epitope selected in the group of epitopes of sequences SEQ ID NO: 60 to SEQ ID NO: 104 and of neoepitopes of sequences SEQ ID NO: 105 to SEQ ID NO: 136.

In some embodiments, said at least one tumor antigen is inserted in a VSV-G comprising SEQ ID NO: 1.

In some embodiments, epitope gp100₄₄₋₅₉ of sequence SEQ ID NO: 71 is inserted in VSV-G comprising, or consisting of, SEQ ID NO: 1. In some embodiments, epitope TRP2₁₈₀₋₁₈₈ of sequence SEQ ID NO: 73 is inserted in VSV-G comprising, or consisting of, SEQ ID NO: 1.

In certain embodiments, the modified vesicular stomatitis virus glycoprotein (VSV-G) for use according to the invention comprises epitope gp100₄₄₋₅₉ of sequence SEQ ID NO: 71 inserted in VSV-G comprising, or consisting of, SEQ ID NO: 1 and epitope TRP2₁₈₀₋₁₈₈ of sequence SEQ ID NO: 73 inserted in VSV-G comprising, or consisting of, SEQ ID NO: 1.

In some embodiments, antigens and/or epitopes according to the invention are inserted into VSV-G by recombinant DNA methods. Nucleic acids of the present invention can be readily prepared by the skilled person using techniques known in the art (e.g., see Sambrook et al., Molecular Cloning: A Laboratory Manual. New-York: Cold Spring Harbor Laboratory Press, 1989; Ausubel et al., Short Protocols in Molecular Biology. New-York: John Wiley and Sons, 1992). In practice, the modified sequence of VSV-G may be obtained by artificial gene synthesis. This allows an adaptation of codon usage for a better expression of the sequence (Angov et al., 2011. Biotechnol. J. 6(6):650-659). The optimized sequence may be subcloned into an expression vector. In another example, a synthetic nucleic acid sequence or vector containing a nucleic acid sequence encoding an antigen and/or an epitope to be inserted into VSV-G may be specifically designed to include restriction endonuclease sites matched to a specified endonuclease-cut nucleic acid sequence encoding VSV-G or to a specified endonuclease-cut nucleic acid sequence previously added into the VSV-G sequence. Where a desirable VSV-G insertion site contains a single, unique restriction endonuclease site, the antigen's and/or the epitope's nucleic acid sequence may be preferably engineered to include matched restriction sites at both ends of the sequence. In this manner, the sequence encoding the antigen and/or the epitope may be inserted into the VSV-G sequence without removal of any VSV-G-encoding nucleotides. Care is taken to match the antigen-encoding and/or the epitope-encoding nucleic acid sequence to be inserted with the reading frame of the VSV-G sequence so that normal expression of the encoded VSV-G with the encoded antigen and/or epitope of interest is achieved. Modified VSV-G can also result from Gibson assembly cloning where multiple DNA fragments can be assembled, regardless of fragment length or end compatibility.

In some embodiments, the at least one antigen, or a fragment thereof, is inserted into VSV-G at any VSV-G permissive insertion site, preferably at a VSV-G permissive epitope insertion site.

In some embodiments, the at least one antigen, or a fragment thereof, is inserted into VSV-G, in highly variable regions. In some embodiments, said highly variable regions are defined on the basis of sequence alignments of VSV-G from various strains. These highly variable regions can undergo sequence modifications without affecting the stability and/or function of the polypeptide. In some embodiments, said highly variable regions are regions which are exposed at the surface of the resulting polypeptide. In some embodiments, said highly variable regions are regions comprised in exposed turns, including α-turns, β-turns, γ-turns, δ-turns, π-turns, ω-turns, loops and/or hairpins. Suitable regions for inserting the at least one antigen, or a fragment thereof, can be determined by methods known from the skilled person, using for example protein structure prediction software and/or loop modeling software.

In some embodiments, the at least one antigen, or a fragment thereof, is inserted into VSV-G, at its C-terminal extremity, i.e., after the last amino acid residue of its sequence.

In some embodiments, the at least one antigen, or a fragment thereof, is inserted into VSV-G from vesicular stomatitis Indiana virus (VSIV) (SEQ ID NO: 1) within region(s) selected from the group consisting of:
Region 1: amino acid residues 1 to 19 of SEQ ID NO: 1;
Region 2: amino acid residues 42 to 61 of SEQ ID NO: 1;
Region 3: amino acid residues 184 to 233 of SEQ ID NO: 1;
Region 4: amino acid residues 253 to 268 of SEQ ID NO: 1;
Region 5: amino acid residues 270 to 289 of SEQ ID NO: 1;
Region 6: amino acid residues 362 to 372 of SEQ ID NO: 1; and
Region 7: after amino acid residue 511, i.e., at the C-terminal extremity of SEQ ID NO: 1.

In one embodiment, the at least one antigen, or a fragment thereof, is inserted into VSV-G from vesicular stomatitis New Jersey virus (VSNJV) (SEQ ID NO: 2) within region(s) selected from the group consisting of:
Region 1: amino acid residues 1 to 19 of SEQ ID NO: 2;
Region 2: amino acid residues 42 to 61 of SEQ ID NO: 2;
Region 3: amino acid residues 184 to 233 of SEQ ID NO: 2;
Region 4: amino acid residues 253 to 272 of SEQ ID NO: 2;
Region 5: amino acid residues 274 to 293 of SEQ ID NO: 2;
Region 6: amino acid residues 366 to 376 of SEQ ID NO: 2; and
Region 7: after amino acid residue 517, i.e., at the C-terminal extremity of SEQ ID NO: 2.

In one embodiment, the at least one antigen, or a fragment thereof, is inserted into VSV-G from Chandipura virus (CHPV) (SEQ ID NO: 3) within region(s) selected from the group consisting of:
Region 1: amino acid residues 1 to 24 of SEQ ID NO: 3;
Region 2: amino acid residues 47 to 66 of SEQ ID NO: 3;
Region 3: amino acid residues 189 to 237 of SEQ ID NO: 3;
Region 4: amino acid residues 257 to 276 of SEQ ID NO: 3;
Region 5: amino acid residues 278 to 297 of SEQ ID NO: 3;
Region 6: amino acid residues 370 to 381 of SEQ ID NO: 3; and
Region 7: after amino acid residue 530, i.e., at the C-terminal extremity of SEQ ID NO: 3.

In one embodiment, the at least one antigen, or a fragment thereof, is inserted into VSV-G from Cocal virus (COCV) (SEQ ID NO: 4) within region(s) selected from the group consisting of:
Region 1: amino acid residues 1 to 20 of SEQ ID NO: 4;
Region 2: amino acid residues 43 to 62 of SEQ ID NO: 4;
Region 3: amino acid residues 185 to 234 of SEQ ID NO: 4;
Region 4: amino acid residues 254 to 269 of SEQ ID NO: 4;
Region 5: amino acid residues 271 to 290 of SEQ ID NO: 4;
Region 6: amino acid residues 363 to 373 of SEQ ID NO: 4; and
Region 7: after amino acid residue 512, i.e., at the C-terminal extremity of SEQ ID NO: 4.

In one embodiment, the at least one antigen, or a fragment thereof, is inserted into VSV-G from Piry virus (PIRYV) (SEQ ID NO: 5) within region(s) selected from the group consisting of:
Region 1: amino acid residues 1 to 21 of SEQ ID NO: 5;
Region 2: amino acid residues 44 to 63 of SEQ ID NO: 5;
Region 3: amino acid residues 186 to 233 of SEQ ID NO: 5;
Region 4: amino acid residues 253 to 272 of SEQ ID NO: 5;
Region 5: amino acid residues 274 to 293 of SEQ ID NO: 5;
Region 6: amino acid residues 366 to 377 of SEQ ID NO: 5; and
Region 7: after amino acid residue 529, i.e., at the C-terminal extremity of SEQ ID NO: 5.

In one embodiment, the at least one antigen, or a fragment thereof, is inserted into VSV-G from Isfahan virus (ISFV) (SEQ ID NO: 6) within region(s) selected from the group consisting of:
Region 1: amino acid residues 1 to 23 of SEQ ID NO: 6;
Region 2: amino acid residues 46 to 65 of SEQ ID NO: 6;
Region 3: amino acid residues 188 to 236 of SEQ ID NO: 6;
Region 4: amino acid residues 256 to 275 of SEQ ID NO: 6;
Region 5: amino acid residues 277 to 296 of SEQ ID NO: 6;
Region 6: amino acid residues 369 to 380 of SEQ ID NO: 6; and
Region 7: after amino acid residue 523, i.e., at the C-terminal extremity of SEQ ID NO: 6.

In one embodiment, the at least one antigen, or a fragment thereof, is inserted into VSV-G from Spring viraemia of carp virus (SVCV) (SEQ ID NO: 7) within region(s) selected from the group consisting of:
Region 1: amino acid residues 1 to 20 of SEQ ID NO: 7;
Region 2: amino acid residues 44 to 63 of SEQ ID NO: 7;
Region 3: amino acid residues 186 to 235 of SEQ ID NO: 7;
Region 4: amino acid residues 254 to 270 of SEQ ID NO: 7;
Region 5: amino acid residues 272 to 291 of SEQ ID NO: 7;
Region 6: amino acid residues 364 to 374 of SEQ ID NO: 7; and
Region 7: after amino acid residue 509, i.e., at the C-terminal extremity of SEQ ID NO: 7.

In one embodiment, the at least one antigen, or a fragment thereof, is inserted into VSV-G from Alagoas virus (VSAV) (SEQ ID NO: 8) within region(s) selected from the group consisting of:
Region 1: amino acid residues 1 to 20 of SEQ ID NO: 8;
Region 2: amino acid residues 43 to 62 of SEQ ID NO: 8;
Region 3: amino acid residues 185 to 234 of SEQ ID NO: 8;
Region 4: amino acid residues 254 to 269 of SEQ ID NO: 8;
Region 5: amino acid residues 271 to 290 of SEQ ID NO: 8;
Region 6: amino acid residues 363 to 373 of SEQ ID NO: 8; and
Region 7: after amino acid residue 511, i.e., at the C-terminal extremity of SEQ ID NO: 8.

In one embodiment, the at least one antigen, or a fragment thereof, is inserted into VSV-G from Carajás virus (CJSV) (SEQ ID NO: 9) within region(s) selected from the group consisting of:
Region 1: amino acid residues 1 to 24 of SEQ ID NO: 9;
Region 2: amino acid residues 47 to 66 of SEQ ID NO: 9;
Region 3: amino acid residues 189 to 238 of SEQ ID NO: 9;
Region 4: amino acid residues 258 to 277 of SEQ ID NO: 9;
Region 5: amino acid residues 279 to 298 of SEQ ID NO: 9;
Region 6: amino acid residues 371 to 381 of SEQ ID NO: 9; and
Region 7: after amino acid residue 523, i.e., at the C-terminal extremity of SEQ ID NO: 9.

In one embodiment, the at least one antigen, or a fragment thereof, is inserted into VSV-G from Maraba virus (MARAV) (SEQ ID NO: 10) within region(s) selected from the group consisting of:
Region 1: amino acid residues 1 to 19 of SEQ ID NO: 10;
Region 2: amino acid residues 42 to 61 of SEQ ID NO: 10;
Region 3: amino acid residues 184 to 233 of SEQ ID NO: 10;
Region 4: amino acid residues 253 to 268 of SEQ ID NO: 10;
Region 5: amino acid residues 270 to 289 of SEQ ID NO: 10;
Region 6: amino acid residues 362 to 372 of SEQ ID NO: 10; and
Region 7: after amino acid residue 512, i.e., at the C-terminal extremity of SEQ ID NO: 10.

In another embodiment, the at least one antigen, or a fragment thereof, is inserted into VSV-G from a virus strain classified or provisionally classified in the Vesiculovirus genus such as Chandipura virus (CHPV), Cocal virus (COCV), Indiana virus (VSIV), Isfahan virus (ISFV), New Jersey virus (VSNJV), Piry virus (PIRYV), Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURY), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viraemia of carp virus (SVCV), Tupaia virus (TUPV), Ulcerative disease rhabdovirus (UDRV) and Yug Bogdanovac virus (YBV). The at least one antigen, or a fragment thereof, is inserted in positions that are readily selected by the one skilled in the art.

As used hereafter, and unless indicated otherwise, the positions into which the antigen(s), or a fragment thereof, is/are inserted are defined by the amino acid residue directly after the insertion site. In other words, insertion position 18 corresponds to the region between amino acid residues 17 and 18.

In some embodiments, the at least one antigen, or a fragment thereof, is inserted into VSV-G from vesicular stomatitis Indiana virus (VSIV) (SEQ ID NO: 1) at a VSV-G amino acid position selected from the group comprising or consisting of positions 13, 14, 15, 16, 17, 18, 19, 20, 21, 22, 23, 46,47, 48, 49, 50, 51, 52, 53, 54, 55, 56, 57, 58, 59, 60, 186, 187, 188, 189, 190, 191, 192, 193, 194, 195, 196, 197, 198, 199, 200, 201, 202, 212, 213, 214, 215, 216, 217, 218, 219, 220, 221, 222, 363, 364, 365, 366, 367, 368, 369, 370, 371, 372, 373 and C-terminal extremity, and combinations thereof.

In certain embodiments, the at least one antigen, or a fragment thereof, is inserted into VSV-G from vesicular stomatitis Indiana virus (VSIV) (SEQ ID NO: 1) at a VSV-G amino acid position selected from the group comprising or consisting of positions 18, 51, 55, 191, 196, 217, 368 and C-terminal extremity, and combinations thereof.

In a preferred embodiment, the at least one antigen, or a fragment thereof, is inserted into VSV-G at VSV-G amino acid positions 18 and/or 191 of SEQ ID NO: 1. In other words, in a preferred embodiment, the nucleic acid sequence encoding the at least one antigen, or a fragment thereof, is inserted into the nucleic acid sequence encoding VSV-G such that the expressed modified VSV-G will include the antigen inserted at VSV-G amino acid position 18 and/or 191 of SEQ ID NO: 1.

In another preferred embodiment, the at least one antigen, or a fragment thereof, is inserted into VSV-G at the C-terminal extremity of VSV-G.

In a particular embodiment, more than one antigen, or a fragment thereof, is inserted into VSV-G at VSV-G amino acid positions 18 of SEQ ID NO: 1. In a particular embodiment, more than one antigen, or a fragment thereof, is inserted into VSV-G at VSV-G amino acid positions 51 of SEQ ID NO: 1. In a particular embodiment, more than one antigen, or a fragment thereof, is inserted into VSV-G at VSV-G amino acid positions 55 of SEQ ID NO: 1. In a particular embodiment, more than one antigen, or a fragment thereof, is inserted into VSV-G at VSV-G amino acid positions 191 of SEQ ID NO: 1. In a particular embodiment, more than one antigen, or a fragment thereof, is inserted into VSV-G at VSV-G amino acid positions 196 of SEQ ID NO: 1. In a particular embodiment, more than one antigen, or a fragment thereof, is inserted into VSV-G at VSV-G amino acid positions 217 of SEQ ID NO: 1. In a particular embodiment, more than one antigen, or a fragment thereof, is inserted into VSV-G at VSV-G amino acid positions 368 of SEQ ID NO: 1. In a particular embodiment, more than one antigen, or a fragment thereof, is inserted into VSV-G at VSV-G C-terminal extremity.

Techniques to determine amino acid positions in a VSV-G other than VSV-G from vesicular stomatitis Indiana virus (VSIV) (SEQ ID NO: 1) into which at least one epitope or fragment thereof can be inserted are well-known in the art.

In some embodiments, multiple antigen, or a fragment thereof, may be inserted into VSV-G, e.g., at more than one site in VSV-G, preferably at two or more sites. In some embodiments, the modified VSV-G of the invention comprises multiple copies of the same antigen, or a fragment thereof. In another embodiment, the modified VSV-G of the invention comprises one copy of different antigen, or a fragment thereof. In still another embodiment, the modified VSV-G of the invention comprises one or more copies of different antigens, or fragments thereof.

In some embodiments, epitope gp100₄₄₋₅₉ of sequence SEQ ID NO: 71 is inserted at VSV-G amino acid positions 18 of SEQ ID NO: 1. In some embodiments, epitope TRP2₁₈₀₋₁₈₈ of sequence SEQ ID NO: 73 is inserted at VSV-G amino acid positions 191 of SEQ ID NO: 1.

In certain embodiments, the modified vesicular stomatitis virus glycoprotein (VSV-G) for use according to the invention comprises epitope gp100₄₄₋₅₉ of sequence SEQ ID NO: 71 inserted at VSV-G amino acid positions 18 of SEQ ID NO: 1 and/or epitope TRP2₁₈₀₋₁₈₈ of sequence SEQ ID NO: 73 inserted at VSV-G amino acid positions 191 of SEQ ID NO: 1.

In some embodiments, the modified vesicular stomatitis virus glycoprotein (VSV-G) for use according to the invention comprises, consists essentially of, or consists of a polypeptide of sequence SEQ ID NO: 138.

A second aspect of the invention relates to a nucleic acid sequence encoding a modified vesicular stomatitis virus glycoprotein (VSV-G) according to the invention for use in preventing and/or treating a brain tumor in an individual in need thereof.

The invention also relates to a nucleic acid sequence encoding the modified vesicular stomatitis virus glycoprotein (VSV-G) according to the invention for use in preventing and/or treating a brain tumor.

In some embodiments, said nucleic acid sequence is to be administered before a surgery in said individual as to remove all or part of the tumor, in particular a tumor resection.

An “encoding sequence” or a sequence “encoding” a modified VSV-G, is meant to refer to a nucleotide sequence that, when expressed, results in the production of that modified VSV-G, i.e., the nucleotide sequence encodes an amino acid sequence for that modified VSV-G. In some embodiments, the encoding sequence includes a start codon (usually ATG) and a stop codon.

In certain embodiments, the nucleic acid sequence is an isolated an isolated nucleic acid sequence.

In some embodiments, nucleic acids according to the invention may be obtained by conventional methods well known to those skilled in the art. Typically, said nucleic acid is a DNA or RNA molecule, which may be included in a suitable vector, such as a plasmid, cosmid, episome, artificial chromosome, phage or viral vector.

In certain embodiments, the nucleic acid according to the invention is a DNA molecule. In another embodiment, the nucleic acid according to the invention is a RNA molecule. In a particular embodiment, the nucleic acid according to the invention is a mRNA molecule.

In some embodiments, the codon usage bias of the nucleic acid according to the invention is optimized. As used herein, the term “codon usage bias” refers to the high-frequency preferential use of a particular codon (as opposed to other, synonymous codons) encoding (or coding for) an amino acid within a given organism, tissue or cell. A codon usage bias may be expressed as a quantitative measurement of the rate at which a particular codon is used in the genome of a particular organism, tissue or cell, for example, when compared to other codons that encode the same amino acid. Various methods are known to those of skill in the art for determining codon usage bias. In some embodiments, codon usage bias may be determined by the codon adaptation index (CAI) method, which is essentially a measurement of the distance of a gene's codon usage to the codon usage of a predefined set of highly-expressed genes (Sharp and Li, 1987. Nucleic Acids Res. 15:1281-95). Alternative methods for determining a codon usage bias include MILC (Measure Independent of Length and Composition) (Supek and Vlahovicek, 2005. BMC Bioinformatics. 6:182) and relative synonymous codon usage (RSCU), which is the observed frequency of a particular codon divided by the frequency expected from equal usage of all the synonymous codons for that amino acid (Sharp et al., 1986. Nucleic Acids Res. 14:5125-43). RSCU values close to 1.0 indicate a lack of bias for the particular codon, whereas departure from 1.0 reflects codon usage bias.

Techniques for nucleic acid manipulation are well known. Reagents useful in applying such techniques, such as restriction enzymes and the like, are widely known in the art and commercially available from a number of suppliers.

In some embodiments, the nucleic acid encoding the modified vesicular stomatitis virus glycoprotein (VSV-G) for use according to the invention comprises, consists essentially of, or consists of a nucleic acid of sequence SEQ ID NO: 137.

As used herein, a brain tumor according to the invention includes primary tumors and metastatic tumors. In some embodiments, a primary brain tumor may be selected in a group comprising a glioblastoma, an anaplastic astrocytoma, a meningioma, and an oligodendroglial tumor. In certain embodiments, the primary brain tumor is a glioblastoma. In some embodiments, the metastatic brain tumor is resulting from metastasis of a primary tumor selected in a group comprising a bladder tumor, a bone tumor, a breast tumor, a tumor of the cervix, a tumor of the upper aero digestive tract, a colorectal tumor, an endometrial tumor, a germ cell tumor, a Hodgkin lymphoma, a kidney tumor, a laryngeal tumor, a leukemia, a liver tumor, a lung tumor, a myeloma, a nephroblastoma (Wilms tumor), a non-Hodgkin lymphoma, an esophageal tumor, an osteosarcoma, an ovarian tumor, a pancreatic tumor, a pleural tumor, a prostate tumor, a retinoblastoma, a skin tumor (including a melanoma), a small intestine tumor, a soft tissue sarcoma, a stomach tumor, a testicular tumor and a thyroid tumor. In certain embodiments, the metastasis of a primary tumor is selected in a group comprising a lung tumor, a melanoma, a kidney tumor, a breast tumor, a colorectal tumor, and an ovarian tumor.

The invention further relates to a vector containing a nucleic acid sequence encoding a modified VSV-G according to the invention for use in preventing and/or treating a brain tumor in an individual in need thereof.

The invention further relates to a vector containing a nucleic acid sequence encoding a modified VSV-G according to the invention for use in preventing and/or treating a brain tumor.

In certain embodiments, said vector is to be administered before a surgery in said individual as to remove all or part of the tumor, in particular a tumor resection.

As used herein, the vector allows expressing a nucleic acid sequence encoding a modified VSV-G according to the invention, and may therefore comprise suitable elements for controlling transcription, such as, e.g., promoter(s), enhancer(s) and, optionally, terminator(s); and, optionally translation.

The present invention also relates to the recombinant vectors into which a nucleic acid sequence according to the invention is inserted. Suitable recombinant vectors may, e.g., be cloning vectors, or expression vectors.

The terms “vector”, “cloning vector” and “expression vector” refer to the vehicle by which the nucleic acid sequence of the invention may be introduced into a host cell, so as to transform the host and promote expression (e.g., transcription and translation) of the polynucleotide.

Any expression vector for animal cell may be used, as long as a nucleic acid sequence encoding a modified VSV-G according to the invention can be inserted and expressed.

Examples of suitable vectors include, but are not limited to, pVAX2, pAGE107, pAGE103, pHSG274, pKCR, pSG1 β d2-4 and the like.

Other examples of vectors include replicating plasmids comprising an origin of replication, or integrative plasmids, such as for instance pUC, pcDNA, pBR, and the like.

In some embodiments, the vector may not comprise a gene encoding antibiotic resistance. In practice, selection may be based either on the complementation of auxotrophic strain, toxin-antitoxin systems, operator-repressor titration, RNA markers, or on the overexpression of a growth essential gene. Minicircles or any other method that allow removing of the antibiotic resistance gene from the initial vector can also be used (Vandermeulen et al., 2011. Mol. Ther. 19(11):1942-49).

In some embodiments, the nucleic acid according to the invention may be ligated into an expression vector which has been specifically optimized for nucleic acid-based vaccination. Elements include, but are not limited to, a transcriptional promoter, immunogenic epitopes, additional cistrons encoding immunoenhancing or immunomodulatory genes (such as ubiquitin), with their own promoters, transcriptional terminator, bacterial origin of replication, antibiotic resistance gene or another selection marker, and CpG sequences to stimulate innate immunity, all of which are well known to those skilled in the art. Optionally, the vector may comprise internal ribosome entry sites (IRES).

In some embodiments, the vector may comprise tissue-specific promoters or enhancers to limit expression of the nucleic acid to a particular tissue type, e.g., the brain and/or the spinal cord.

In some embodiments, tissue- or cell-specific promoters may be used to target the expression of the modified VSV-G to antigen-presenting cells.

Examples of other eukaryotic transcription promoters include, but are not limited to, the Rous sarcoma virus (RSV) promoter, the simian virus 40 (SV40) promoter, the human elongation factor-1 α (EF-1α) promoter and the human ubiquitin C (UbC) promoter.

Suitable vectors include any plasmid DNA construct comprising a nucleic acid of the invention, operatively linked to a eukaryotic promoter. Examples of such vectors include the pCMV series of expression vectors, commercially available from Stratagene® (La Jolla, Calif.); the pcDNA or pREP series of expression vectors by Invitrogen® Corporation (Carlsbad, Calif.).

In certain embodiments, the vector is a viral vector. In some embodiments, suitable viral vectors include, but are not limited to, adenoviral, retroviral, herpes virus and AAV vectors. Such recombinant viral vectors may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, and the like. Detailed protocols for producing such replication-defective recombinant viruses may be found for instance in WO1995014785, WO1996022378, U.S. Pat. Nos. 5,882,877, 6,013,516, 4,861,719, 5,278,056 and WO1994019478.

In some embodiments, the vectors may also contain nucleic acid sequences encoding selected class I and class II MHC molecules, costimulation and other immunoregulatory molecules, ABC transporter proteins, including the TAP1 and TAP2 proteins.

In some embodiments, the vectors may also contain at least one positive marker that enables the selection of dendritic cells carrying the inserted nucleic acids.

Another aspect of the invention relates to a dendritic cell population transfected by the nucleic acid sequence encoding the modified VSV-G or a vector according to the invention for use in preventing and/or treating a brain tumor in an individual in need thereof.

In one aspect, the invention relates to a dendritic cell population transfected by the nucleic acid sequence encoding the modified VSV-G or a vector according to the invention for use in preventing and/or treating a brain tumor.

In some embodiments, said dendritic cell population is to be administered before a surgery in said individual as to remove all or part of the tumor, in particular a tumor resection.

Another object of the invention is a dendritic cell population transfected by a nucleic acid sequence or a vector according to the invention.

In some embodiments, one or more nucleic acids are inserted ex vivo into dendritic cells, such that one or more selected antigen(s), are presented in effective amounts on the surface of the dendritic cells. By “effective amount” is meant that presentation is sufficient to enable the dendritic cells to provoke an immune response.

Nucleic acids encoding the desired antigens, for presentation in the dendritic cells are preferably recombinant expression vectors in which high levels of expression may occur.

Expression of the nucleic acids of interest after transfection into dendritic cells may be confirmed by immunoassays or biological assays. For example, expression of introduced nucleic acids into cells may be confirmed by detecting the binding to the cells of labeled antibodies specific for the antigens of interest using assays well known in the art such as FACS (Fluorescent Activated Cell Sorting) or ELISA (enzyme-linked immunoabsorbent assay) or simply by staining (e.g., with β-gal) and determining cell counts.

T cell activation may be detected by various known methods, including measuring changes in the proliferation of T cells, killing of target cells, tetramer staining, and secretion of certain regulatory factors, such as lymphokines, expression of mRNA of certain immunoregulatory molecules, or a combination of these.

A still further aspect of the invention relates to a composition comprising a modified VSV-G, a nucleic acid sequence, a vector or a dendritic cell population according to the invention for use in preventing and/or treating a brain tumor in an individual in need thereof.

In one aspect, the invention relates to a composition comprising a modified VSV-G, a nucleic acid sequence, a vector or a dendritic cell population according to the invention for use in preventing and/or treating a brain tumor.

In certain embodiments, said composition is to be administered before a surgery in said individual as to remove all or part of the tumor, in particular a tumor resection.

In some embodiments, the invention relates to composition consisting essentially of, or consisting of, a modified VSV-G, a nucleic acid sequence, a vector or a dendritic cell according to the invention for use in preventing and/or treating a brain tumor in an individual in need thereof.

In certain embodiments, the invention relates to a composition consisting essentially of, or consisting of, a modified VSV-G, a vector or a dendritic cell population according to the invention for use in preventing and/or treating a brain tumor.

In some embodiments, said composition is to be administered before a surgery in said individual as to remove all or part of the tumor, in particular a tumor resection.

As used herein, the expression “consist essentially of” is intended to mean that the composition to which it refers does not comprise any other active ingredient, i.e., an ingredient responsible for a physiologic or therapeutic response, other than the modified VSV-G, the nucleic acid sequence, the vector or dendritic cell population according to the invention.

The present invention further relates to a pharmaceutical composition comprising, consisting essentially of, or consisting of, a modified VSV-G, a nucleic acid sequence, a vector or a dendritic cell population according to the invention and at least one pharmaceutically acceptable excipient, for use in preventing and/or treating a brain tumor.

In some embodiments, said pharmaceutical composition is to be administered before a surgery in said individual as to remove all or part of the tumor, in particular a tumor resection.

As used herein, the term “pharmaceutical composition” includes veterinary composition.

The present invention also relates to an immunogenic composition comprising, consisting essentially of, or consisting of, a modified VSV-G, a nucleic acid sequence, a vector or a dendritic cell population according to the invention, for use in preventing and/or treating a brain tumor.

In certain embodiments, said immunogenic composition is to be administered before a surgery in said individual as to remove all or part of the tumor, in particular a tumor resection.

In another aspect, the invention relates to a vaccine comprising a modified VSV-G, a nucleic acid sequence, a vector, or a dendritic cell population according to the invention, and optionally at least one adjuvant, for use in preventing and/or treating a brain tumor in an individual in need thereof.

In certain aspects, the invention relates to a vaccine comprising a modified VSV-G, a nucleic acid sequence, a vector, or a dendritic cell population according to the invention, and optionally at least one adjuvant, for use in preventing and/or treating a brain tumor.

In some embodiments, said vaccine is to be administered before a surgery in said individual as to remove all or part of the tumor, in particular a tumor resection.

In some embodiments, the vaccine according to the invention is a prophylactic vaccine.

As used herein, the expression “prophylactic vaccine” is intended to refer to a vaccine that is to be administered before definitive clinical signs, diagnosis or identification of a brain tumor. According to this embodiment, the vaccine is to be administered to prevent a brain tumor.

If the vaccine appears to induce an effective, but short-lived immune response, prophylactic vaccines may also be designed to be used as booster vaccines. Such booster vaccines are given to individuals who have previously received a vaccination, with the intention of prolonging the period of protection.

In some embodiments, the vaccine according to the invention is a therapeutic vaccine.

As used herein, the expression “therapeutic vaccine” is intended to refer to a vaccine that is to be administered after first clinical signs, diagnosis or identification of the disease. According to this embodiment, the vaccine is to be administered to treat a brain tumor.

In some embodiments, said vaccine is a nucleic acid vaccine or a protein vaccine.

In certain embodiments, the vaccine is a nucleic acid vaccine Immunization with nucleic acid may also be referred to as “genetic immunization”, “RNA immunization” or “DNA immunization”.

Accordingly, in some embodiments, the vaccine according to the invention comprises a nucleic acid sequence encoding a modified VSV-G according to the invention. In some embodiments, the nucleic acid sequence is a DNA nucleic acid sequence. In some alternative embodiments, the nucleic acid sequence is a RNA nucleic acid sequence.

In some embodiment, the vaccine according to the invention may express more than one modified VSV-G. Illustratively, the vaccine according to the invention may express two modified VSV-G or more. In a particular embodiment, the vaccine of the invention may express two modified VSV-G or more, wherein said modified VSV-G are distinct.

According to this embodiment, the nucleic acid vaccine according to the invention may comprise two nucleic acid sequences each encoding a distinct modified VSV-G.

In a preferred embodiment, the vaccine of the invention expresses a first modified VSV-G and a second modified VSV-G wherein the first modified VSV-G comprises a CD8 T cell epitope and wherein the second modified VSV-G comprises a CD4 T cell epitope.

In some embodiments, said first and/or second modified VSV-G, nucleic acid sequence, vector, composition, cell or vaccine may further comprise a universal antigenic CD4 T cell epitope or nucleic acid sequence thereof.

In another embodiment, the vaccine according to the invention is a protein vaccine. Accordingly, in some embodiments, the vaccine according to the invention comprises a modified VSV-G according to the invention. In another embodiment, the vaccine according to the invention comprises two modified VSV-G or more. In a particular embodiment, the vaccine according to the invention comprises two modified VSV-G or more, wherein said modified VSV-G are distinct.

In a preferred embodiment, the vaccine according to the invention comprises a first modified VSV-G and a second modified VSV-G wherein the first modified VSV-G comprises a CD8 T cell epitope and wherein the second modified VSV G comprises a CD4 T cell epitope.

In some embodiments, the vaccine according to the present invention is used in a prime-boost strategy to induce robust and long-lasting immune response to the antigen. Priming and boosting vaccination protocols based on repeated injections of the same antigenic construct are well known and result in strong CTL responses. In general, the first dose may not produce protective immunity, but only “primes” the immune system. A protective immune response develops after the second or third dose.

In certain embodiments, the vaccine according to the invention is used in a conventional prime-boost strategy, in which the same vaccine is to be administered to the individual in multiple doses. In a preferred embodiment, the vaccine is used in one or more inoculations. These boosts are performed according to conventional techniques, and can be further optimized empirically in terms of schedule of administration, route of administration, choice of adjuvant, dose, and potential sequence when administered with another vaccine, therapy or homologous vaccine.

In another embodiment, the vaccine according to the present invention is used in a prime-boost strategy using an alternative administration of modified VSV-G comprising xenoantigen and autoantigen or fragment thereof, or of polynucleotides encoding modified VSV-G comprising xenoantigen and autoantigen or fragment thereof. Specifically, according to the present invention, the individual is first treated, or “primed”, with a vaccine encoding an antigen of foreign origin (a “xenoantigen”), or a fragment thereof. Subsequently, the individual is then treated with another vaccine encoding an antigen or fragment thereof which is corresponding to the xenoantigen, but is of self-origin (“autoantigen”). This way, the immune reaction to the antigen is boosted. The boosting step may be repeated one or more times.

In some embodiments, vaccines of the present invention are formulated so as to comprise one or more pharmaceutically acceptable carriers or excipients such as water, saline, dextrose, glycerol, and the like, as well as combinations thereof. In some embodiments, vaccines may also contain auxiliary substances such as wetting agents, emulsifying agents, buffers, adjuvants, and the like.

In some embodiments, the excipient for use in the nucleic acid vaccines according to the present invention may be a polymer such as a cationic polymer or a non-ionic polymer, including but not limited to, polyoxyethylene (POE), polyoxypropylene (POP), polyethyleneglycol (PEG), linear or branched polyethylenimine (PEI). In certain embodiments, polymers can form block copolymers, for instance, a POE-POP-POE block copolymer. As used herein, the term “polyplex” refers to polymer-nucleic acid or copolymer-nucleic acid complexes.

In certain embodiments, the nucleic acid vaccines may be formulated so as to comprise cationic lipids. Optionally, lipids can be mannosylated. As used herein, the term “lipoplex” refers to lipid-nucleic acid or liposome-nucleic acid complexes.

In some embodiments, lipoplexes may further be complexed with polymers or copolymers to form tertiary complexes. These tertiary complexes may have enhanced in vivo delivery and transfection capacities of the nucleic acid to the targeted cells, and thereby, facilitate enhanced immune responses.

In certain embodiments, suitable carriers for use in the nucleic acid vaccines of the present invention may be nanoparticles. These include but are not limited to: nano-emulsions, dendrimers, nano-gold, lipid-based nanoparticles, liposomes, drug-carrier conjugates, antibody-drug complexes, and magnetic nanoparticles.

In some embodiments, the nucleic acid vaccine of the present invention may be formulated so as to comprise one or more adjuvants, which may increase its immunogenicity. A skilled artisan is capable of identifying suitable adjuvants that may increase the immune response of the nucleic acid vaccines according to the present invention in comparison to administration of a non-adjuvanted nucleic acid vaccine.

In some embodiments, the adjuvant is selected from the group consisting of α-interferon, γ-interferon, platelet derived growth factor (PDGF), TNF-α, TNF-β, GM-CSF, epidermal growth factor (EGF), HIV-1 gag, cutaneous T cell-attracting chemokine (CTACK), epithelial thymus-expressed chemokine (TECK), mucosae-associated epithelial chemokine (MEC), IL-2, IL-12, IL-15, IL-28, MHC, CD80, CD86 including IL-15 having the signal sequence deleted and optionally including the signal peptide from IgE. Other genes which may be useful adjuvants include those encoding: MCP-I, MIP-loc, MIP-I p, IL-8, RANTES, L-selectin, P-selectin, E-selectin, CD34, GIyCAM-1, MadCAM-1, LFA-I, VLA-I, Mac-1, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, Inactive NIK, SAP K, SAP-I, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2, functional fragments and combinations thereof.

In some preferred embodiments, the adjuvant is selected from the group consisting of α-interferon, γ-interferon, IL-2, IL-8, IL-12, IL-15, IL-18, IL-28, MCP-I, MIP-Ia, MIP-Ip, RANTES, RANK, RANK LIGAND, Ox40, Ox40 LIGAND, CTACK, TECK, MEC, functional fragments and combinations thereof.

In some preferred embodiments, the adjuvant is selected from the group consisting of α-interferon, γ-interferon, IL-2, IL-12, functional fragments and combinations thereof.

In another embodiment, adjuvant for use in the nucleic acid vaccines according to the present invention may comprise a mineral-based compound, such as one or more forms of an aluminum phosphate-based adjuvant, or one or more forms of a calcium phosphate.

In another embodiment, adjuvant may be saponin, monophosphoryl lipid A or any other compound that can be used to increase immunogenicity of the nucleic acid vaccine.

In some embodiments, the nucleic acid vaccine according to the present invention is formulated so as to comprise one or more genetic adjuvants which may increase immunogenicity of the nucleic acid vaccines according to the present invention. It is within the purview of the skilled artisan to utilize available genetic adjuvants which may increase the immune response of the nucleic acid vaccines according to the present invention in comparison to administration of a non-adjuvanted nucleic acid vaccine.

As used herein, genetic adjuvants refer to immunomodulatory molecules encoded by a plasmidic vector. They stimulate the innate immune system to trigger appropriate dendritic cell maturation and thereby a robust, specific, and long-lasting adaptive immune response Immunomodulatory molecules include cytokines, chemokines, or immune stimulatory molecules, such as toll-like receptor agonists or interferon regulatory factors.

Examples of other adjuvants include, but are not limited to, particle bombardment using DNA-coated or RNA-coated gold beads; co-administration of polynucleotide vaccines with plasmid DNA expressing cytokines, chemokines, or costimulatory molecules.

In some embodiments, the composition, the vector, the dendritic cell population or the vaccine according to the invention may be administered ex vivo or in vivo.

According to the present invention, an effective administration protocol (i.e., administering a composition or vaccine in an effective manner) comprises suitable dose parameters and modes of administration that result in eliciting an immune response in an individual with a brain tumor, or that is at risk of developing a brain tumor, preferably so that the individual is protected from the brain tumor.

Effective dose parameters can be determined using methods standard in the art for brain tumors. Such methods include, but are not limited to, determination of survival rates, side effects (i.e., toxicity) and progression or regression of a brain tumor.

In particular, the effectiveness of dose parameters of a therapeutic composition of the present invention when treating a brain tumor may be determined by assessing response rates. Such response rates refer to the percentage of treated individuals in a population of individuals that respond with either partial or complete remission. Remission can be determined by, for example, measuring tumor size, e.g., by imagery analysis, such as e.g., MRI, PET-scan and the likes.

According to the present invention, a suitable single dose size is a dose that is capable of eliciting an antigen-specific immune response in a subject when administered once or more times over a suitable time period. Doses can vary depending upon the size, weight, gender, the age, the general physical condition, the severity and/or the stage of the brain tumor. In the treatment of brain tumors, a therapeutic effective amount can be dependent upon whether the tumor being treated is a primary tumor or a metastatic form of cancer. In practice, one of skills in the art can readily determine prophylactic or therapeutic effective amounts for administration based on the weight of a subject and the route of administration.

In some embodiments, a prophylactic or therapeutic effective amount of the composition or vaccine of the invention is from about 0.5 pg to about 5 mg per kilogram body weight of the individual being administered the composition or vaccine. In a preferred embodiment, a prophylactic or therapeutic effective amount of the composition or vaccine of the invention is from about 0.1 μg to about 1 mg per kilogram body weight of the individual, preferably from about 1 μg to about 100 μg per kilogram body weight of the individual, preferably from about 10 μg to about 75 μg per kilogram body weight of the individual, preferably about 50 μg per kilogram body weight of the subject.

Within the scope of the invention, the expression “from about 0.5 pg to about 5 mg” encompasses 0.5 pg, 0.75 pg, 1 pg, 1.5 pg, 2 pg, 2.5 pg, 5 pg, 7.5 pg, 10 pg, 20 pg, 25 pg, 50 pg, 75 pg, 100 pg, 250 pg, 500 pg, 750 pg, 1 ng, 1 ng, 1.5 ng, 2 ng, 2.5 ng, 5 ng, 7.5 ng, 10 ng, 20 ng, 25 ng, 50 ng, 75 ng, 100 ng, 250 ng, 500 ng, 750 ng, 1 μg, 1.5 μg, 2 μg, 2.5 μg, 5 μg, 7.5 μg, 10 μg, 20 μg, 25 μg, 50 μg, 75 μg, 100 μg, 250 μg, 500 μg, 750 μg, 1 mg, 2 mg, 2.5 mg and 5 mg.

When T cells or dendritic cells are administered to an individual with brain tumor, the cells may be administered (with or without adjuvant) parenterally (including, e.g., intravenous, intraperitoneal, intramuscular, intradermal, and subcutaneous administration). Alternatively, the cells may be administered locally by direct injection into a tumor.

Adjuvants include any known pharmaceutically acceptable carrier. Parenteral vehicles for use as pharmaceutical carriers include, but are not limited to, sodium chloride solution, Ringer's dextrose, dextrose and sodium chloride, and lactated Ringer's. Other adjuvants may be added as desired such as antimicrobials.

As an example, T cells may be administered by intravenous infusion, at doses of about 10⁸ to 10⁹ cells/m² of body surface area (see, e.g., Ridell et al., 1992. Science. 257:238-241). Infusion can be repeated at desired intervals, for example, monthly. Recipients are monitored during and after T cell infusions for any evidence of adverse effects.

According to a preferred embodiment, the T cells are obtained from the same individual from whom the dendritic cells were obtained.

According to another embodiment, the T cells are obtained from an individual and the dendritic cells, which are used to stimulate the T cells, are obtained from an HLA-matched healthy donor (e.g., a sibling), or vice versa.

According to yet another embodiment, both the T cells and the dendritic cells are obtained from an HLA-matched healthy donor. This embodiment may be particularly advantageous, for example, when the individual is a late stage cancer patient who has been treated with radiation and/or chemotherapy agents and may not be able to provide sufficient or efficient dendritic or T cells.

According to another embodiment of the invention, dendritic cells isolated from a subject are cultured, transfected in vitro and administered back to the subject to stimulate an immune response, including T cell activation. As such, the dendritic cells constitute a vaccine and/or immunotherapeutic agent.

As an example, dendritic cells presenting antigen are administered, via intravenous infusion, at a dose of, for example, about 10⁵ to 10⁹ cells, more preferably from about 10⁶ to about 10⁷ dendritic cells per administration. According to an embodiment, dendritic cells presenting antigen are administered at a dose from about 5×10⁶ to about 5×10⁸ dendritic cells per administration, preferably from about 10⁷ to about 2×10⁸ dendritic cells per administration. Within the scope of the invention, the expression “10⁵ to 10⁹ cells” encompasses 10⁵, 5×10⁵, 10⁶, 5×10⁶, 10⁷, 5×10⁷, 10⁸, 5×10⁸ and 10⁹ cells.

In some embodiments, infusion can be repeated at desired intervals based upon the subject's immune response.

When vaccines of the invention are used in a prime-boost strategy, “boosters” of the vaccine are preferably administered when the immune response against the peptide, preferably antigen, as wanted or as needed to provide an immune response or induce a memory response against a particular antigen. Boosters can be administered from about 1 week to several years after the original administration. In some embodiments, an administration schedule is one in which from about 0.5 pg to about 5 mg of a vaccine per kilogram body weight of the subject is to be administered from about one to about 4 times over a time period of from about 1 month to about 6 months.

It will be obvious to one of skills in the art that the number of doses administered to a subject is dependent upon the extent of the disease and the response of said subject to the treatment. For example, a large tumor may require more doses than a smaller tumor. In some cases, however, an individual having a large tumor may require fewer doses than as individual with a smaller tumor, if the individual with the large tumor responds more favorably to the composition or vaccine than the individual with the smaller tumor. Thus, it is within the scope of the present invention that a suitable number of doses includes any number required to treat a given disease.

In some embodiments, said modified VSV-G, vector, dendritic cell population or vaccine is to be administered in combination with another tumor treatment.

In some embodiments, the other tumor treatment is a brain tumor treatment, preferably a glioblastoma treatment.

In the treatment of tumors, the modified VSV-G, nucleic acid, vector, composition, cell population or vaccine of the invention is to be administered before, and optionally after, surgical resection of a tumor from the individual.

In practice, the method of the invention may be combined with further prophylactic and/or therapeutic approaches to enhance the efficacy of the method.

In another embodiment, the modified VSV-G, nucleic acid sequence, vector, composition, cell population or vaccine of the invention may be administered in combination with another therapeutic molecule, such as chemotherapeutic agents, anti-angiogenesis agents, checkpoint blockade antibodies or other molecules that reduce immune-suppression; or in combination with another antitumor treatment, such as radiation therapy, hormonal therapy, targeted therapy or immunotherapy.

In a particular embodiment, the modified VSV-G, nucleic acid sequence, vector, composition, cell or vaccine of the invention may be administered in combination with antibodies. Examples of antibodies which may be co-administered include, but are not limited to, antibodies anti-PD-1 (e.g., nivolumab, pidilizumab and MK-3475), antibodies anti-PD-L1 (e.g., BMS-936559, MEDI4736 and MPDL33280A), antibodies anti-CTLA4 (e.g., ipilimumab and tremelimumab), antibodies anti-OX40, antibodies anti-4-1BB, antibodies anti-CD47, antibodies anti-KIR, antibodies anti-CD40, antibodies anti-LAG-3 and combinations thereof. In some embodiments, the antibodies are selected in a group comprising ipilimumab, pembrolizumab, nivolumab, atezolizumab, avelumab and durvalumab.

In a particular embodiment, the modified VSV-G, nucleic acid sequence, vector, composition, cell or vaccine of the invention may be administered in combination with stimulating factors. Example of stimulating factors which may be co-administered include, but are not limited to, granulocyte-macrophage colony-stimulating factor (GM-CSF) (e.g., sargramostim or molgramostim).

In some embodiments, the further tumor treatment is a treatment targeting a primary tumor, which metastasis resulted in a brain tumor.

Another object of the present invention is a method for inducing in an individual a protective immune response comprising administering a modified VSV-G, nucleic acid sequence, vector, composition, cell or vaccine of the invention to an individual in need thereof.

In one embodiment, the method of the invention is for inducing in an individual a protective immune response against cancer.

In certain embodiments, said modified VSV-G is to be administered before said further tumor treatment.

In some embodiments, said modified VSV-G, nucleic acid sequence, vector, dendritic cell population or vaccine is to be administered at least once to the individual before said other tumor treatment.

In some embodiments, the surgery encompasses tumor resection. Within the scope of the invention the expression “tumor resection” is intended to refer to the surgical removal, at least in part, of the tumor.

In certain embodiments, said other tumor treatment is a tumor resection.

Without wishing to be bound to a theory, the inventors consider that the tumor resection may allow reducing the number of tumor cells and may allow inducing a local inflammation that could strengthen the adaptive immunity activated by the vaccine. In addition, they consider that the vaccine may allow activating the host adaptive immune system against the residual tumor cells, thus avoiding brain tumor recurrences.

In practice, tumor resection may be performed by surgery by a medical professional following the standard and good practices.

In some embodiments, the tumor resection is a brain tumor resection, in particular a glioblastoma resection.

In certain embodiments, the chemotherapy comprises at least one anti-cancer compound, in particular an anti-cancer compound selected in a group comprising an alkylating agent, a purine analogue, a pyrimidine analogue, an anthracycline, bleomycin, mitomycin, an inhibitor of topo-isomerase 1, an inhibitor of topo-isomerase 2, a taxan, a monoclonal antibody, a cytokine, an inhibitor of a protein kinase, and the like, an anti-inflammatory agent, a radical scavenger, an immunomodulatory agent or any other drug acting on the tumor resection microenvironment.

In some embodiments, the chemotherapy comprises temozolomide. In certain embodiments, the targeted drug therapy comprises bevacizumab.

In some embodiments, said modified VSV-G, nucleic acid sequence, vector, dendritic cell population or vaccine is to be administered to the individual by intramuscular injection, intradermal injection, intra-tumoral injection, peritumoral injection, gene gun, electroporation or sonoporation.

In some aspects, the invention relates to a modified vesicular stomatitis virus glycoprotein (VSV-G) comprising at least one tumor antigen, or a fragment thereof, for use for ameliorating the prognostic of an individual with brain tumor. Similarly, the invention relates to the use of a modified vesicular stomatitis virus glycoprotein (VSV-G) comprising at least one tumor antigen, or a fragment thereof, for ameliorating the prognostic of an individual with a brain tumor.

In some aspects, the invention relates to the use of a modified vesicular stomatitis virus glycoprotein (VSV-G) comprising at least one tumor antigen, or a fragment thereof, for preventing and/or treating a brain tumor in an individual in need thereof.

Another aspect of the invention relates to the use of a modified vesicular stomatitis virus glycoprotein (VSV-G) comprising at least one tumor antigen, or a fragment thereof, for preventing and/or treating a brain tumor.

A still further aspect of the invention relates to the use of a modified vesicular stomatitis virus glycoprotein (VSV-G) comprising at least one tumor antigen, or a fragment thereof, for the manufacture or the preparation of a medicament for preventing and/or treating a brain tumor.

In certain embodiments, said modified VSV-G is to be administered before a surgery in said individual as to remove all or part of the tumor, in particular a tumor resection.

One aspect of the invention relates to a method for preventing and/or treating a brain tumor in an individual in need thereof, comprising the administration of a therapeutically effective amount of a modified vesicular stomatitis virus glycoprotein (VSV-G) comprising at least one tumor antigen, or a fragment thereof.

One aspect of the invention relates to a method for preventing and/or treating a brain tumor in an individual in need thereof, comprising the steps of:
a) administering a therapeutically effective amount of a modified vesicular stomatitis virus glycoprotein (VSV-G) comprising at least one tumor antigen, or a fragment thereof; and
b) performing a surgery as to remove all or part of the tumor, in particular a tumor resection.

In one aspect, the invention relates to a method for preventing and/or treating a brain tumor in an individual in need thereof, comprising the administration of a therapeutically effective amount of a vector comprising a modified VSV-G according to the invention, a dendritic cell population comprising a modified VSV-G according to the invention, or a vaccine composition comprising a modified VSV-G according to the invention.

In one aspect, the invention relates to a method for preventing and/or treating a brain tumor in an individual in need thereof, comprising the steps of:
a) administering a therapeutically effective amount of a vector comprising a modified VSV-G according to the invention, a dendritic cell population comprising a modified VSV-G according to the invention, or a vaccine composition comprising a modified VSV-G according to the invention; and
b) performing a surgery as to remove all or part of the tumor, in particular a tumor resection.

One aspect of the invention relates to a method for preventing and/or treating a brain tumor in an individual in need thereof, comprising the administration of a therapeutically effective amount of a modified VSV-G, a vector, a dendritic cell population or a vaccine according to the invention, in combination with another tumor treatment.

A further aspect of the invention relates to a method for preventing and/or treating a brain tumor in an individual in need thereof, comprising the steps of:
a) administering a therapeutically effective amount of a modified VSV-G, a vector, a dendritic cell population or a vaccine according to the invention, in combination with a further tumor treatment; and
b) performing a surgery as to remove all or part of the tumor, in particular a tumor resection.

In some embodiments, the further tumor treatment is administered before, during or after the administration of a therapeutically effective amount of a modified VSV-G, a nucleic acid sequence, a vector, a dendritic cell population or a vaccine according to the invention.

In certain embodiments, the other tumor treatment is a tumor resection.

A still further aspect of the invention relates to a method for preventing and/or treating a brain tumor in an individual in need thereof, comprising the steps of:
a) administering to said individual a therapeutically effective amount of a modified VSV-G, a nucleic acid sequence, a vector, a dendritic cell population or a vaccine according to the invention;
b) performing a surgery in said individual as to remove all or part of the tumor, in particular a brain tumor resection.

In practice, the surgery is a tumor resection, in particular a brain tumor resection, in particular a glioblastoma resection.

In another aspect, the invention also relates to a method for ameliorating the prognostic of an individual with brain tumor, comprising the steps of:
a) administering to said individual a therapeutically effective amount of a modified VSV-G, a nucleic acid sequence, a vector, a dendritic cell population or a vaccine according to the invention; and
b) optionally, performing a surgery in said individual as to remove all or part of the tumor, in particular a brain tumor resection.

In a further aspect, the invention also relates to a method for ameliorating the prognostic of an individual with brain tumor, comprising the steps of:
a) administering to said individual a therapeutically effective amount of a modified VSV-G, a nucleic acid sequence, a vector, a dendritic cell population or a vaccine according to the invention; and
b) performing a surgery in said individual as to remove all or part of the tumor, in particular a brain tumor resection.

In some embodiments, the methods disclosed hereinabove may comprise a step of:
c) administering to said individual a second therapeutically effective amount of a modified VSV-G, a nucleic acid sequence, a vector, a dendritic cell population or a vaccine according to the invention.

### BRIEF DESCRIPTION OF THE DRAWINGS

FIGS. 1A-C is a combination of plots showing a therapeutic immunization with pTOP vaccines in a murine glioblastoma model. FIG. 1A: Schematic protocol. C57BL/6 mice were first subcutaneously injected with GL261 tumor cells (2×10⁶ cells). pTOP7 vaccine (1 μg) was intramuscularly electroporated 2, 9 and 16 days after the injection of tumor cells. FIG. 1B: Evolution of tumor volume expressed in mm³ (ordinate), as a function of time expressed in day (abscissa). FIG. 1C: Survival curves, as the percent of survival (ordinate) as a function of time expressed in day (abscissa). The errors bars represent mean±SEM; n=6-7. Statistical analysis: Two-way ANOVA with Bonferroni post-tests or Mantel-Cox test for comparison of survival curves. *p value<0.05 as compared to naive.

FIGS. 2A-G is a combination of plots showing a therapeutic immunization and resection in an orthotopic glioblastoma model and evaluation of the systemic immune response. FIG. 2A: Schematic protocol. C57BL/6 mice first received an intracranial injection of 5×10⁴ cells of GL261 at day 0. MRI was used to monitor brain tumor at day 10 and 27. pTOP7 vaccine (1 μg) was intramuscularly electroporated 16, 23 and 29 days after the injection of tumor cells and resection of the tumor was performed at day 17, n=9-12. FIG. 2B: Survival curves for the therapeutic immunization.

FIG. 2C: Representative axial T2-weighted MRI image of an untreated mouse brain before (day 10) and after tumor resection (day 27). The white arrows indicate the GL261 primary and recurrent tumor, respectively. FIG. 2D-G: Analysis of immune cells in the spleen, 29 days after GL261 inoculation. The percentage of splenic CD8 is shown for all the groups (FIG. 2D) and the production of IFNγ from splenocytes stimulated with TRP2 peptide is assessed by ELISPOT (FIG. 2E). The percentage of MDSC (FIG. 2F) and the ratio of M1/M2 macrophages (FIG. 2G) are displayed. The errors bars represent mean±SEM; n=7-9. Statistical analysis: One-way ANOVA with Tukey multiple comparisons test or Mantel-Cox test for comparison of survival curves. *p value<0.05 and **p value<0.01 as compared to naive or to the specified group.

FIGS. 3A-G are a combination of plots showing the evaluation of immune cells and immunosuppressive cells in the brain, 29 days after GL261 inoculation. FIG. 3A-B: Total number of CD8 and ratio of IFNγ secreting CD8/total CD8, respectively. FIG. 3C-D: Total number of CD4 and ratio of IFNγ secreting CD4/total CD4, respectively. FIG. 3E-F: percentage of MDSC and ratio of M1/M2 macrophages in the brain, respectively. FIG. 3G: Number of Treg in the brain. The errors bars represent mean±SEM; n=7-9. Statistical analysis: One-way ANOVA with Tukey multiple comparisons test. *p value<0.05, **p value<0.01, ***p value<0.001 as compared to naive or to the specified group.

### EXAMPLES

The present invention is further illustrated by the following examples.

### Example 1

1. Materials and Methods

### 1.1—Materials

a) Plasmids and Primers

pTOP refers to the plasmids encoding VSV-G (of sequence SEQ ID NO: 1) in which the foreign epitopes were inserted. Codon-optimized gene sequences were designed using GeneOptimizer and obtained by standard gene synthesis from GeneArt® (Thermo Fisher Scientific®, US). The sequences were subcloned in the pVAX2 vector using cohesive-end cloning. To allow easy modifications of the epitopes, several restriction sites were added. Digestion by BamHI and HindIII or by SpeI and EcoRI allows insertion in position (18) or (191), respectively. The inserted epitopes are detailed in Table 4.

TABLE 4
Plasmids details

| Name | Position (18) | Position (191) |
|---|---|---|
| pVAX2-VSVG | — | — |
| pTOP7¹ | gp100₄₄₋₅₉ (WNRQLYPEWTEAQRLD²) | TRP2₁₈₀₋₁₈₈ (SVYDFFVWL³) |

¹The resulting construct, VSVG-gp100₄₄₋₅₉-TRP2₁₈₀₋₁₈₈, has a nucleic acid sequence SEQ ID NO: 137; and an amino acid sequence SEQ ID NO: 138;
²Amino acid sequence SEQ ID NO: 71;
³Amino acid sequence SEQ ID NO: 73.

The positions into which the epitopes are inserted are defined by the amino acid residue directly after the insertion site. In other words, insertion position (18) corresponds to the region between amino acid residues 17 and 18. For the epitopes inserted at the N terminus (e.g., position (18), just after the signal peptide), an additional lysine residue was included. Overlapping phosphorylated oligonucleotides that encoded the restricted epitope (IDT-DNA®, Belgium) were incorporated in the digested vector using cohesive-end cloning. The plasmids were prepared using the EndoFree Plasmid Mega or Giga Kit (Qiagen®, Germany) and diluted in PBS. The quality of the purified plasmid was assessed by the ratio of optical densities and by 1% agarose gel electrophoresis. Plasmids were sequenced by Sanger DNA sequencing (Genewiz®, UK) and stored at −20° C.

Primers used herein are depicted in Table 5 below:

TABLE 5
Primers used herein

| Gene | | |
|---|---|---|
| length | Primer sequence (5′→3′) | Amplicon |
| TRP2 | For-CCAGGATGACCGTGAGCAA (SEQ ID NO: 139) | 171 bp |
| | Rev-GGGCAGTCAGGGAATGGAT (SEQ ID NO: 140) | |
| Murine- | For-GGAGCTTCCTTCCCGTGCTT (SEQ ID NO: 141) | 321 bp |
| gp100 | Rev-GCTCCCATTGATGATGGTGT (SEQ ID NO: 142) | |
| Human- | For-ATAGGTGCTTTGCTGGCTGT (SEQ ID NO: 143) | 263 bp |
| gp100 | Rev-ACCTGCCCATCTGGCAATAC (SEQ ID NO: 144) | |
| VSV-G | For-AACTGGCACAACGACCTGAT (SEQ ID NO: 145) | 144 bp |
| | Rev-GATGTACTTGGGGCCGTACC (SEQ ID NO: 146) | |
| b-actin | For-ACTCCTATGTGGGTGACGAG (SEQ ID NO: 147) | 206 bp |
| | Rev-CATCTTTTCACGGTTGGCCTTAG | |
| | (SEQ ID NO: 148) | |

b) Cell lines

GL261 tumor cells were cultured in DMEM. Media were supplemented with 10% FBS, 100 μg/mL streptomycin, and 100 U/mL penicillin (Gibco®, Life Technologies®, USA). Cells were sub-cultured in 75 cm² culture flasks (Corning® T-75, Sigma-Aldrich, USA) and incubated at 37° C. and 5% CO₂.

c) Animals

Six- to eight-week-old C57BL/6NRj, Balb/c and DBA/2 female mice were obtained from Janvier Labs® (France) and housed in an air-conditioned animal facility with ad libitum access to food and water. Temperature and humidity were monitored daily. For tumor implantation and electroporation, the mice were anaesthetized with a 150 to 200 μL intraperitoneal injection of 10 mg/mL ketamine and 1 mg/mL xylazine. All in vivo experiments were performed following the Belgian national regulation's guidelines in accordance with EU Directive 2010/63/EU, and were approved by the ethical committee for animal care of the faculty of medicine of the Université Catholique de Louvain (2011/UCL/MD/007, 2014/UCL/MD/004 and 2016/UCL/MD/001).

### 1.2—Methods

a) Immunization

Intramuscular electroporation—After the mouse hair was removed using a rodent shaver (AgnTho's, Lidingö, Sweden), 30 μL of a PBS solution containing 1 μg of plasmid was injected into the tibial cranial muscle. The leg was placed between 4-mm-spaced plate electrodes, and 8 square-wave electric pulses (200 V/cm, 20 ms, 2 Hz) were delivered. For prophylactic immunizations, two boosts were similarly applied two and four weeks after priming. For therapeutic immunizations, the vaccine was administered 2, 9 and 16 days after subcutaneous tumor injection or 16, 23 and 29 days after orthotopic injection.

For all electroporation protocols, electric pulses were generated by a Gemini System generator and delivered with BTX Caliper Electrodes (BTX; both from VWR International, Belgium). A conductive gel was used to ensure electrical contact with the skin (Aquasonic 100; Parker Laboratories®, Inc., USA).

b) Subcutaneous Tumor Implantation and Tumor Measurement

A total of 2×10⁶ GL261 cells diluted in 100 μl of PBS were injected subcutaneously into the right flank of C57Bl/6 mice. The tumor cells were inoculated before the plasmid treatment for the therapeutic experiments and two weeks after complete immunization for the prophylactic studies. Tumor size was measured three times a week with an electronic digital caliper. Tumor volume was calculated as the length×width×height (in mm³). Mice were sacrificed when the tumor volume was greater than 1500 mm³ or when they reached the end points (behavior changes e.g. lack of grooming and clinical signs of distress e.g.: paralysis, arched back, lack of movement plus 10% body weight loss and/or 20% body weight loss).

c) Orthotopic GL261 Brain Tumor (Glioblastoma) Syngeneic Model

Mice were anesthetized by intraperitoneal injection of ketamine/xylazine (100 mg/kg and 13 mg/kg, respectively) and fixed in a stereotactic frame. A surgical high-speed drill (Vellman®, Belgium) was used to perform a hole in the right frontal lobe and 5×10⁴ GL261 cells were slowly injected using a Hamilton syringe fitted with a 26S needle. To obtain cortical tumors, the injection coordinates were 0.5 mm posterior, 2.1 mm lateral from the bregma and 2.2 mm deep from the outer border of the cranium. The presence, volume and location of the tumors were determined by magnetic resonance imaging (MRI), which was performed for all mice included in the study before the surgical resection of the tumor. Animals presenting GL261 tumors were randomly divided into four groups.

d) Magnetic Resonance Imaging

MRI was performed using a 11.7 T Bruker Biospec MRI system (Bruker®, Germany) equipped with a 1H quadrature transmit/receive surface cryoprobe after anesthetizing animals with isoflurane mixed with air (2.5% for induction, 1% for maintenance). Tumor was visualized using rapid acquisition with relaxation enhancement (RARE) sequence (repetition time=2500 ms; effective echo time=30 ms; RARE factor=8; field of view=2×2 cm; matrix 256×256; Slice thickness=0.3 mm; 25 contiguous slices were acquired, N average=4).

e) Surgical Resection of the Tumor Mass

At day 17 post-tumor inoculation, the tumor mass was surgically removed using the biopsy-punch resection technique. Briefly, animals were anaesthetized with ketamine/xylazine and immobilized in a stereotactic frame. An 8 mm incision was made in the midline along the previous surgical scar and a 2.1 mm diameter circular cranial window was created around the previous burr hole using fine tip tweezers (Dumont®, Switzerland) to expose the brain. A 2 mm diameter biopsy punch (Kai Medical®, Germany) was then inserted 3 mm deep and twisted for 15 s to cut the brain region surrounding the tumor. Once withdrawn, the tumor and brain tissues were aspired using a diaphragm vacuum pump (Vaccubrand® GBMH+CO KG, Germany) connected to a Pasteur pipette and a 200 μl tip. Residual blood was removed from the surgical cavity using a hemostatic triangle (Fine Science Tools®, Germany) The cranial window was then sealed with a 4×4 mm square piece of Neuro-Patch® (Aesculap®, Germany) impregnated with a reconstituted fibrin hydrogel (25 mg/mL fibrin, 10 IU/mL thrombin, equal volumes; Baxter Innovations®, Austria).

All animals were monitored daily and an MRI follow-up was performed 27 days after surgery. Eight to nine animals per group were sacrificed 29 days post-tumor inoculation for immunological analysis (FACS and PCR). The spleen and the brain of the animals were collected for further analysis. The remaining animals were sacrificed when they reached the end points.

f) Flow Cytometry Analysis of Immune Cells

TAM, MDSC, CD4 and CD8 T cell populations in brains and spleens removed 29 days after GL261 orthotopical cell injection were analyzed by FACS. Cells were passed through a 70 μm cell strainer (BD Falcon®, New Jersey), collected, counted using an automatic cell counter (Invitrogen®, California) and washed with PBS, before adding the blocking solution with anti-CD16/CD32 antibody for 10 minutes on ice (clone 93, Biolegend®, San Diego, Calif.). Cells were washed and incubated for 60 minutes at 4° C. with the following antibodies: anti-CD3-APC-Cy7 (Biolegend®, San Diego, Calif.), anti-CD4-PE (BD Bioscience®, United Kingdom), anti-CD8-BV421 (Biolegend®, San Diego, Calif.) for CD4 and CD8 T cell detection; with anti-CD11b-FITC (BD Bioscience®, United Kingdom), anti-F4/80-AF647 (BD Bioscience®, United Kingdom), anti-CD206-BV421 (Biolegend®, San Diego, Calif.) and anti-Grl-PE (BD bioscience, United Kingdom) for TAMs and MDSCs; with anti-CD3-APC-Cy7, anti-CD8-FITC (Proimmune®, United Kingdom) and Pentamers-TRP2-PE (Proimmune®, United Kingdom) for the detection of TRP-2-specific CD8 T cells. For staining with antiFoxP3-AF488 (BD Bioscience®, United Kingdom) or anti-IFNg-APC (Biolegend®, San Diego, Calif.), cells were previously incubated overnight at 4° C. with a permeabilization/fixation solution (eBioscience™ Foxp3/Transcription Factor Staining Buffer Set, Thermo Fisher, Waltham, Mass.). Cells were then incubated with anti-CD16/CD32 antibody for 10 minutes on ice (Biolegend®, San Diego, Calif.), washed and incubated for 60 minutes at 4° C. with anti-IFNg-APC or antiFoxP3-AF488 diluted in the permeabilization/fixation solution. Samples were washed with PBS fixed for 10 minutes with 4% formalin and, then, suspended in PBS. Sample data were acquired with FACSVerse (BD Bioscience®, Franklin Lakes, N.J.) and analyzed with FlowJo software (FlowJo® LLC, Ashland, Oregon).

g) Enzyme-Linked ImmunoSpot (ELISpot)

ELISpot was performed according to the manufacturer's instruction (Immunospot, the ELISPOT source, Germany) Briefly, 3×10⁵ fresh splenocytes diluted in 100 μl CTL-Test medium (Immunospot, the ELISPOT source) were cultured overnight at 37° C. in anti-IFNg-coated 96 well plate. For stimulation, 10 ng/μl of TRP2₁₈₀₋₁₈₈ peptide (SVYDFFVWL; SEQ ID NO: 73) was added to the splenocytes and incubated for 2 days. As positive control for splenocyte activation, Cell Stimulation Cocktail (Invitrogen®, California) was used; PBS and a P815 irrelevant peptide (LPYLGWLVF; SEQ ID NO: 149) were used as negative control. The development of the ELISpot plate followed the manufacturer's instruction and pots were counted by using an ELISPOT reader system (the ELISPOT source).

h) RT-PCR Analysis

GL261 cells were analyzed by RT-PCR to verify the presence of TRP2 and gp100 expression. Total RNA was isolated using TRIzol reagent (Thermo Fisher Scientific®, Waltham, Mass.) and phenol separation. The quality and quantity of RNA were evaluated using a nano-spectrophotometer (NanoDrop 2000, Thermo Fisher Scientific®, Waltham, Mass.). One microgram of RNA was reverse transcribed using a first-strand synthesis system (SuperScript™, Thermo Fisher Scientific®, Waltham, Mass.) and oligo(dT) primers according to the supplier's protocol. The resulting cDNA was used as template for 30 cycles of PCR amplification. The PCR products were individualized to electrophoresis on a SYBR Safe (Thermo Fisher Scientific®)-stained 1.5% agarose gel.

i) Statistical Analysis

Statistical analyses were performed using GraphPad Prism 7® for Windows®. P-values lower than 0.05 were considered statistically significant.

2. Results

2.1—Insertion of Tumor Epitopes in the pTOP Plasmid Allows Therapeutic Vaccinations Against Subcutaneous GL261 Tumors

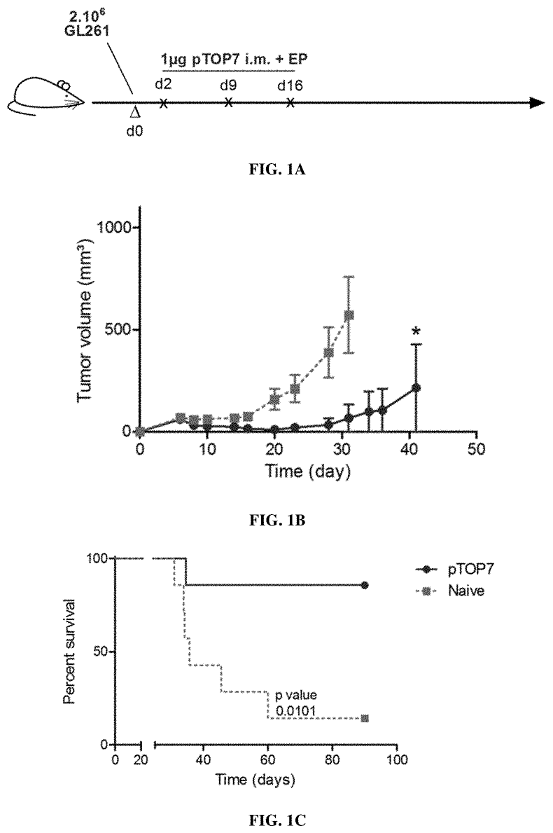

We evaluated the efficacy of pTOP against a brain tumor. pTOP7 was obtained by inserting two tumor epitopes (TRP2₁₈₉₋₁₈₈ and gp100₄₄₋₅₉) in the VSV-G sequence and evaluated as a therapeutic vaccine delivered at days 2, 9 and 16 after subcutaneous tumor cell injection (FIG. 1A). The expression of TRP2 and gp100 in GL261 cells was verified by RT-PCR. Tumor growth of vaccinated mice was significantly delayed compared to the untreated group (FIG. 1B) and 6 out of 7 mice were considered long-term survivors (FIG. 1C). Thus, pTOP7 can be effective against GL261 tumors.

2.2—in a GL261 Orthotopic Model, Tumor Resection and pTOP Vaccination Significantly Prolonged Mice Survival

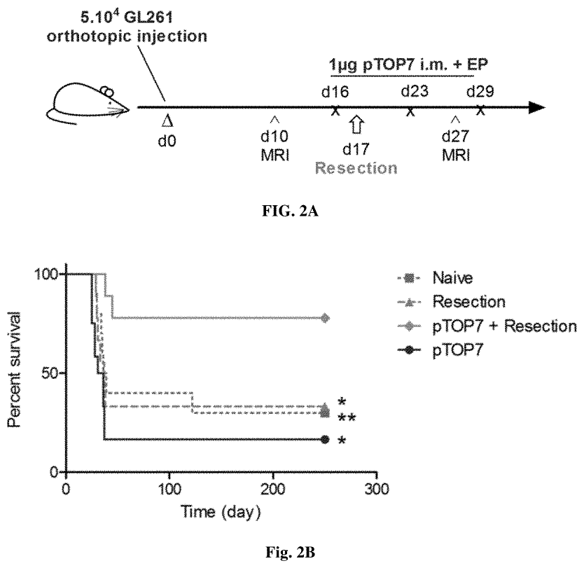

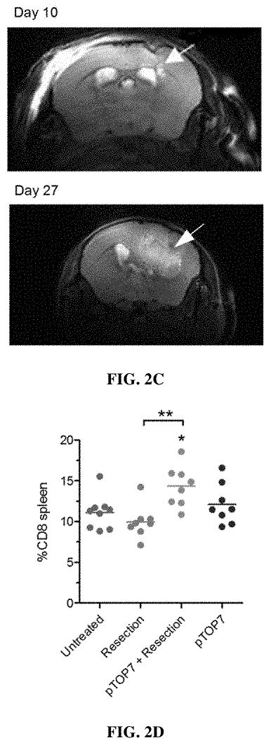

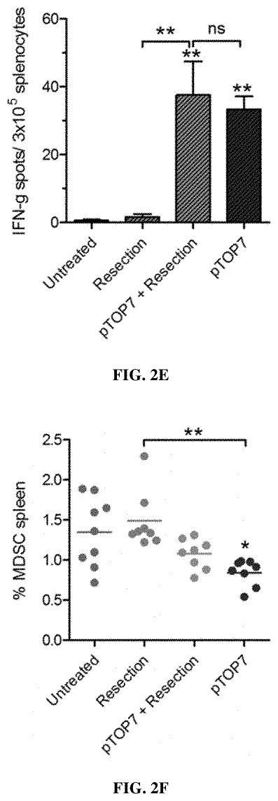

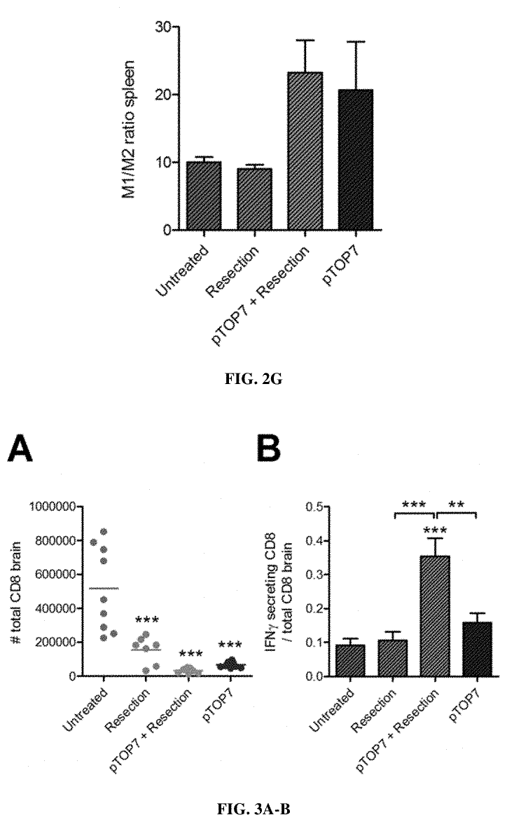

As pTOP7 was highly efficient against the subcutaneously implanted GL261 tumors, we checked whether this vaccine could prevent recurrences in a murine orthotopic GBM model when the first dose of vaccine was administered just before surgical resection. Tumoral lesions of GL261-bearing mice were observed between the cortex and the striatum in all implanted animals at day 10 post-inoculation, by MRI. Mice were vaccinated at day 16, 23 and 29 and the tumor was resected 17 days after the GL261 inoculation (see FIG. 2A). In the control groups (naive, resection or pTOP7), most of the mice showed signs of discomfort and pain starting from day 27-30 after the tumor injection and their median survival time was less than 40 days. However, when resection was combined to therapeutic immunization with pTOP7, 78% of the mice were able to survive for at least 250 days and thus considered as long-term survivors (FIG. 2B). MRI performed 27 days after tumor inoculation confirmed the presence of infiltrative and aggressive recurrences in control groups (FIG. 2C). Due to the infiltrative patterns of the GL261 tumors, we are unable to provide adequate volume estimation of the tumors at the designated time points but the presence of the tumor and of its infiltrative nature was confirmed post-mortem by hematoxylin and eosin staining (H/E).

2.3—pTOP Induced Systemic Antigen-Specific Immune Response and Modulated the Number of Immune Cells in the Spleen

Next, we evaluated the systemic immune activity after resection and/or immunization with pTOP7. To this end, splenocytes were collected 29 days after the tumor challenge and analyzed by flow cytometry and ELISpot. When resection and pTOP7 vaccine were combined, the CD8 infiltration was significantly higher compared to the untreated mice and those that underwent the resection alone (FIG. 2D). The activation of TRP2-specific T cells was assessed by IFNγ ELISpot. In the absence of vaccination, almost no spot has been detected whereas the splenocytes from pTOP7 treated mice showed a significantly higher number of spots (FIG. 2E). In addition, the number of myeloid-derived suppressor cells (MDSC) was lower when mice were treated with pTOP7 (FIG. 2F) and a modulation (albeit not statistically significant) of the M1/M2 macrophage ratio was observed for mice treated with pTOP7 with or without tumor resection (FIG. 2G).

2.4—pTOP and Tumor Resection Enhanced the Activity of Immune Cells and Reduced the Number of Infiltrated Immunosuppressive Cells in the Brain

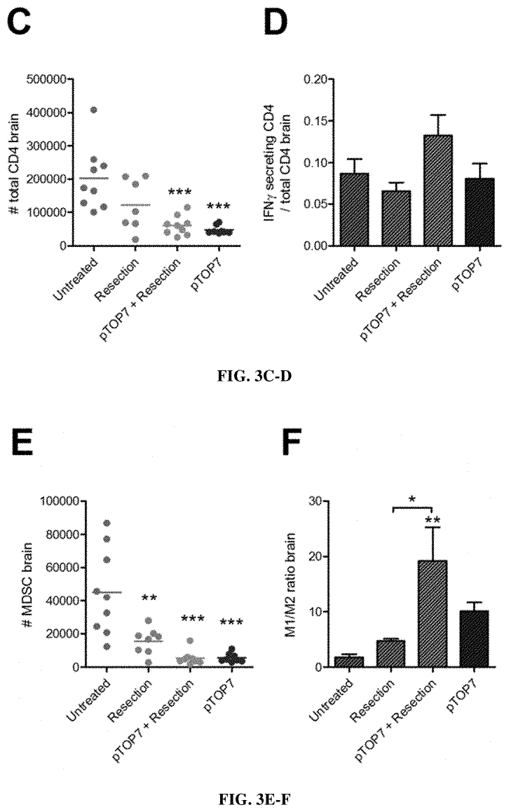

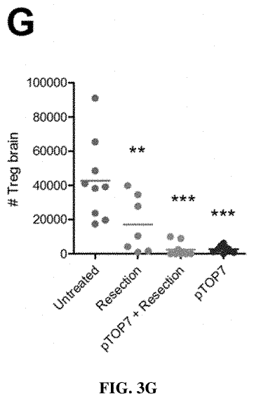

To study the mechanism underpinning the synergy between pTOP7 and the resection of GL261 tumors and their contribution in prolonging mice survival, the infiltration of different immune cells was assessed in the mice brains 29 days after tumor inoculation. A decreased infiltration of CD8 T cells in the treated groups, especially in the combination group (FIG. 3A). However, when analyzing the activity of those cells, we observed that almost 40% of infiltrated CD8 produced IFNγ as compared to only 10-15% for the other groups (FIG. 3B). The same trend was observed for the number of CD4 (FIG. 3C) and IFNγ-secreting CD4 T cells but the slight increase observed in the combination group was not significantly different to the other groups (FIG. 3D). The flow cytometry analysis also revealed a significant decrease of the infiltrated immunosuppressive cells for all the groups compared to the untreated group. This effect was seen for MDSC (FIG. 3E), M1/M2 macrophage ratio (FIG. 3F) and regulatory T cells (FIG. 3G). The greatest on infiltrated immunosuppressive cells was observed when pTOP7 was used alone or in combination with the resection. The M1/M2 macrophage ratio was significantly higher for mice treated with the combination as compared to untreated mice or mice with resected tumors.

3. Discussion

The inventors have demonstrated that pTOP7, i.e. a plasmid encoding a modified VSV-G protein comprising inserted defined T cell epitopes (originating from gp100 and TRP2), was able to generate a specific and long-lasting immune response against GL261 GBM and to target residual GBM cells in mice that had undergone surgical resection. Resection induces immunological changes that could contribute to the vaccine activity such as the induction of excessive healing response, production of inflammatory cytokines and recruitment of both M1 and M2 macrophages. Combined resection and vaccination induced many immunological changes, both systemically and locally. In the brain, the ratio IFNγ-producing CD8/total CD8 was significantly higher, indicating the presence of active infiltrated CD8 T cells. This may indicate that the infiltrated CD8 T cells in the combination group, even if low in number, are not exhausted and still able to recognize the antigen and produce IFNγ. Furthermore, all the vaccinated groups showed higher levels of antigen-specific T cells in the spleen. In addition, less MDSC, M2 macrophages and Treg were observed in the brain suggesting that the immunosuppressive activity was reduced, especially when the vaccine was combined with the resection, thus permitting a higher CD8 T cell activation. The combination of DNA vaccination and surgical resection drastically increased mice survival, due to a decreased infiltration of immunosuppressive cells and the concomitant activity and antigen-specificity of T cells in the brain. The strength of this combination could overcome the limits of each single treatment: from one side the tumor resection reduces the number of tumor cells and induces a local inflammation that could strengthen the adaptive immunity activated by the vaccine. From the other side, the vaccine activates the host adaptive immune system against the residual tumor cells, thus avoiding the GBM recurrences. To the inventors' knowledge, this is the first study reporting the combination between GBM surgical resection and vaccine immunotherapy being performed before the tumor debulking. The vaccine administration prior to surgery might take advantage of the acute inflammatory response induced by the resection to activate specific antitumor immune response acting on residual GBM cells and on the tumor resection microenvironment, thus avoiding the on-set of tumor recurrences.

### Example 2: Assessment of the Combined Efficacy of pTOP Vaccine and Immune Checkpoint Blockade ICB

For orthotopic GBM tumor grafting, C57BL/6 mice were anesthetized and fixed in a stereotactic frame. A surgical high-speed drill (Vellman®, Belgium) was used to perform a hole in the right frontal lobe and 5×10⁴ GL261 cells were slowly injected using a Hamilton syringe fitted with a 26S needle. To obtain cortical tumors, the injection coordinates were 0.5 mm posterior, 2.1 mm lateral from the bregma and 2.2 mm deep from the outer border of the cranium. The presence, volume and location of the tumors were determined by MRI. Animals presenting GL261 tumors were randomly divided into four groups (naive, vaccine alone, ICB alone and combined treatment).

pTOP vaccine—After the mouse hair was removed using a rodent shaver (AgnTho's, Lidingo, Sweden), 30 μL of a PBS solution containing 1 μg of plasmid was injected into the tibial cranial muscle. The leg was placed between 4-mm-spaced plate electrodes, and 8 square-wave electric pulses (200 V/cm, 20 ms, 2 Hz) were delivered. The vaccine was administered 16, 23 and 29 days after GL261 orthotopic injection.

ICB—Immune checkpoint blockade antibodies directed against CTLA4 (clone 9D9) and PD1 (clone 29 F.A12) were purchased from Bioconnect® (Netherlands) and mice were injected intraperitoneally with 100 μg of each antibody in 100 μl of PBS 17, 20 and 23 days after tumor injection.

1-19. (canceled)

20. A method for preventing and/treating a brain tumor in an individual in need thereof, said method comprising the steps of:
a) administering to said individual a therapeutically effective amount of the modified vesicular stomatitis virus glycoprotein (VSV-G) comprising at least one tumor antigen, or a fragment thereof, a nucleic acid encoding the same, a vector containing said nucleic acid, a dendritic cell population transfected by said nucleic acid, a pharmaceutical composition comprising any of the foregoing, or a vaccine comprising any of the foregoing; and
b) performing a surgery in said individual as to remove all or part of the tumor.

21. The method according to claim 20, wherein said surgery is a brain tumor resection.

22. The method according to claim 20, further comprising the step c) of administering to said individual a second therapeutically effective amount of a modified VSV-G, or fragment thereof, nucleic acid, vector, dendritic cell population, pharmaceutical composition or vaccine.

23. The method according to claim 20, wherein said at least one tumor antigen is selected in (i) a group of antigens comprising ALK, GALT3, NA17-A, HSD3B7, BCAN, CHI3L2, CSPG4, FABP7, IGF2BP3, NLGN4X (Neuroligin 4, X-linked), NRCAM, PTPRZ1, TNC, AIM2, gp100, MAGE, TRP2, HER2, IL13Rα2, MAGE A11, SSX5, NOL4, MAGE C2, EPHA2, YKL-40, VEGFR1, VEGFR2, SURVIVIN, pp65, IE1, MART-1, SART-1, HER2/NEU, GNT-V, Tyrosinase, hTERT, B-CYCLIN, IDH1, EGFRvIII, WT-1, HSPPC-96, HB-EGF, EGFR, PCNA, ITGAV, STAT-3, IQGAP-1, HO-1, BSG, SEC61G and PIK3R1, or (ii) a group of neoantigens comprising PAPPA2, NF1, ATP8B3, HOXA1, OR4C3, FAM20B, INSM2, GOLGA6L22, TMEM241, POTEJ, PRKRA, C9orf57, LILRB3, MYLK, ABCA2, ATP1A2, LINC00273, CDH7, ELL, NCAN, TTN, GPR50, LCE1F, GOLGA6L1, GOLGA6L2, LOC645752, DSPP, CRHBP and TENM3.

24. The method according to claim 20, wherein said at least one tumor antigen is selected in (i) a group of antigens comprising gp100, TRP2, pp65 and EGFRvIII, or (ii) a group of neoantigens comprising PAPPA2, NF1, ATP8B3, HOXA1, OR4C3, FAM20B, INSM2, GOLGA6L22, TMEM241, POTEJ, PRKRA, C9orf57, LILRB3, MYLK, ABCA2, ATP1A2, LINC00273, CDH7, ELL, NCAN, TTN, GPR50, LCE1F, GOLGA6L1, GOLGA6L2, LOC645752, DSPP, CRHBP and TENM3.

25. The method according to claim 20, wherein said at least one tumor antigen is gp100 and/or TRP2.

26. The method according to claim 20, wherein said at least one tumor antigen comprises an epitope selected in the group of epitopes of sequences SEQ ID NO: 60 to SEQ ID NO: 104 and of neoepitopes of sequences SEQ ID NO: 105 to SEQ ID NO: 136.

27. The method according to claim 20, wherein said at least one tumor antigen is inserted in a VSV-G comprising SEQ ID NO: 1.

28. The method according to claim 20, wherein said at least one epitope is epitope gp10044-59 of sequence SEQ ID NO: 71 and/or epitope TRP2180-188 of sequence SEQ ID NO: 73.

29. The method according to claim 20, wherein epitope gp10044-59 of sequence SEQ ID NO: 71 is inserted at VSV-G amino acid positions 18 of SEQ ID NO: 1 and/or epitope TRP2180-188 of sequence SEQ ID NO: 73 is inserted at VSV-G amino acid positions 191 of SEQ ID NO: 1.

30. The method according to claim 20, wherein the brain tumor is selected in the group consisting of glioblastoma, anaplastic astrocytoma, meningioma, and oligodendroglial tumor.

31. The method according to claim 20, wherein the brain tumor is a glioblastoma.

32. The method according to claim 20, wherein said modified VSV-G, nucleic acid sequence, vector, dendritic cell population, pharmaceutical composition, or vaccine is administered to the individual by intramuscular injection, intradermal injection, intra-tumoral injection, peritumoral injection, gene gun, electroporation or sonoporation.

33. The method according to claim 20, wherein said pharmaceutical composition comprises at least one pharmaceutically acceptable excipient.

34. The method according to claim 20, wherein said vaccine comprises at least one adjuvant.

35. The method according to claim 20, wherein said vaccine is a nucleic acid vaccine or a protein vaccine.

36. The method according to claim 20, wherein said modified VSV-G, nucleic acid sequence, vector, dendritic cell population, pharmaceutical composition, or vaccine is administered in combination with a further tumor treatment.

37. A method for ameliorating the prognostic of an individual with a brain tumor, said method comprising the steps of:
a) administering to said individual a therapeutically effective amount of the modified vesicular stomatitis virus glycoprotein (VSV-G) comprising at least one tumor antigen, or a fragment thereof, a nucleic acid encoding the same, a vector containing said nucleic acid, a dendritic cell population transfected by said nucleic acid, a pharmaceutical composition comprising any of the foregoing, or a vaccine comprising any of the foregoing; and
b) performing a surgery in said individual as to remove all or part of the tumor.

38. The method according to claim 37, wherein said surgery is a brain tumor resection.

39. The method according to claim 37, wherein the brain tumor is selected in the group consisting of glioblastoma, anaplastic astrocytoma, meningioma, and oligodendroglial tumor.
